(19) United States
(12) Patent Application Publication
(10) Pub. No.: US 2025/0073027 A1
(43) Pub. Date: Mar. 6, 2025

(54) PROSTHETIC VALVE WITH RUNGS OF SUPPORT MEMBERS

(71) Applicant: EDWARDS LIFESCIENCES CORPORATION, Irvine, CA (US)

(72) Inventors: Tamir S. Levi, Zikhron Yaakov (IL); Karin Lavon, Tel-Aviv (IL); Nikolai Gurovich, Hadera (IL)

(21) Appl. No.: 18/949,766

(22) Filed: Nov. 15, 2024

Related U.S. Application Data
(63) Continuation of application No. PCT/US2023/022792, filed on May 18, 2023.
(60) Provisional application No. 63/422,349, filed on Nov. 3, 2022.
(60) Provisional application No. 63/343,986, filed on May 19, 2022.

(51) Int. Cl. A61F 2/24

(52) U.S. Cl. CPC A61F 2/2418; A61F 2210/0057; A61F 2220/0075

(57) ABSTRACT

A prosthetic valve has an annular frame that is radially expandable and a leaflet structure positioned within the frame. The frame has an inflow end, an outflow end, a first row of angled struts defining the outflow end, a second row of angled struts upstream of the first row of angled struts, a third row of angled struts upstream of the second row of angled struts, and a plurality of axial frame members comprising respective first ends and second ends. The first ends are connected to the second row of angled struts and the second ends are connected to the third row of angled struts. The plurality of axial frame members include a plurality of axially extending commissure supports and one or more axial posts located between any two immediately adjacent commissure supports. Each commissure support is configured to support a corresponding commissure of the leaflet structure.

### CROSS-REFERENCE TO RELATED APPLICATIONS

This application is a continuation of International Patent Application No. PCT/US2023/022792, filed May 18, 2023, which claims the benefit of U.S. Provisional Application No. 63/343,986, filed May 19, 2022, and U.S. Provisional Application No. 63/422,349, filed Nov. 3, 2022, all of which applications are hereby incorporated by reference in their entireties.

### FIELD

The present disclosure relates to implantable, radially expandable prosthetic devices, such as prosthetic heart valves, and to methods, assemblies, and apparatuses for delivering, expanding, implanting, and deploying such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

The prosthetic valve can include a radially compressible and expandable frame and a leaflet structure mounted within the frame. In some circumstances, the prosthetic valve can have a sealing member, such as an outer skirt mounted on an outer surface of the frame. The outer skirt can be configured to seal against surrounding native tissue so as to reduce paravalvular leakage past the prosthetic valve.

Despite the recent advancements in percutaneous valve technology, there remains a need for improved transcatheter prosthetic valves, for example, to improve the strength of the frame and/or to reduce the paravalvular leakage.

### SUMMARY

The present disclosure relates to methods and devices for treating valvular diseases. Specifically, the present disclosure is directed to implantable, radially expandable prosthetic devices, such as prosthetic heart valves, and to methods, assemblies, and apparatuses for delivering, expanding, implanting, and deploying such prosthetic devices.

A prosthetic valve can comprise a frame and a valve structure coupled to the frame. In addition to these components, a prosthetic heart valve can further comprise one or more of the components disclosed herein.

In some examples, a frame of a prosthetic valve can comprise an inflow end, an outflow end, a first row of angled struts defining the outflow end, a second row of angled struts being closer to the inflow end than the first row of angled struts, and a plurality of axial frame members bridging the first row of angled struts and the second row of angled struts.

In some examples, a frame of a prosthetic valve can comprise a plurality of support members connecting the plurality of axial frame members.

In some examples, the support members can be narrower than the axial frame members and the first and second rows of angled struts.

In some examples, a frame of a prosthetic valve can comprise a plurality of axial frame members including respective first ends and second ends, the first ends being connected to the first row of angled struts and the second ends being connected to the second row of angled struts.

In some examples, a frame of a prosthetic valve can comprise a plurality of support members connecting the plurality of axial frame members at connecting points located between first ends and second ends of the axial frame members.

In some examples, a prosthetic valve can comprise an outer skirt disposed on an outer surface of the frame.

In some examples, the outer skirt can be connected to the support members.

In some examples, a frame of a prosthetic valve can comprise a first row of angled struts defining the outflow end, a second row of angled struts upstream of the first row of angled struts, a third row of angled struts upstream of the second row of angled struts, and a plurality of axial frame members comprising respective first ends and second ends, the first ends being connected to the second row of angled struts and the second ends being connected to the third row of angled struts.

In some examples, the plurality of axial frame members can comprise a plurality of axially extending commissure supports and one or more axial posts located between any two immediately adjacent commissure supports, each commissure support being configured to support a corresponding commissure of a leaflet structure if the prosthetic valve.

In some examples, a frame of a prosthetic valve can further comprise a fourth row of angled struts upstream of the third row of angled struts.

In some examples, the first row of angled struts and the second row of angled struts define a plurality of first cells of the frame; the second row of angled struts, the third row of angled struts, and the plurality of axial frame members define a plurality of second cells of the frame; and the third row of angled struts and the fourth row of angled struts define a plurality of third cells of the frame.

In some examples, the number of first cells is more than the number of second cells, and the number of second cells equals the number of third cells.

In some examples, the first cells are smaller than the third cells and the third cells are smaller than the second cells when the frame is in the radially expanded configuration.

Certain aspects of the disclosure concern a prosthetic valve. The prosthetic valve can include an annular frame that is radially collapsible to a collapsed configuration and radially expandable to an expanded configuration, and an outer skirt disposed on an outer surface of the annular frame. The annular frame can include an inflow end, an outflow end, a first row of angled struts defining the outflow end, a second row of angled struts being closer to the inflow end than the first row of angled struts, a plurality of axial frame members bridging the first row of angled struts and the second row of angled struts, and a plurality of support members connecting the plurality of axial frame members. The support members can be narrower than the axial frame members and the first and second rows of angled struts. The outer skirt can be connected to the support members.

According to certain aspects of the disclosure, a prosthetic valve can include an annular frame that is radially expandable and compressible, and an outer skirt disposed on an outer surface of the annular frame. The annular frame can include an inflow end, an outflow end, a first row of angled struts defining the outflow end and a second row of angled struts upstream of the first row of angled struts, a plurality of axial frame members including respective first ends and second ends, the first ends being connected to the first row of angled struts and the second ends being connected to the second row of angled struts, and a plurality of support members connecting the plurality of axial frame members at connecting points located between the first ends and the second ends. The outer skirt can be connected to the support members.

Certain aspects of the disclosure also concern an annular frame that is radially expandable and compressible. The annular frame can include an inflow end, an outflow end, a row of outflow struts defining the outflow end, a row of inflow struts defining the inflow end, a row of interconnected struts circumferentially extending between the row of outflow struts and the row of inflow struts, a plurality of axial frame members including respective first ends and second ends, the first ends being connected to the row of outflow struts and the second ends being connected to the row of interconnected struts, and a plurality of support members connecting the plurality of axial frame members and extending between the row of outflow struts and the row of interconnected struts. Each outflow strut can include two angled strut portions interconnected by an apex portion. The support members can be narrower than the axial frame members, the angled strut portions of the outflow struts, and the row of interconnected struts.

According to certain aspects of the disclosure, an annular frame that is radially expandable and compressible can include a row of first end struts defining a first terminal end of the annular frame, a row of second end struts defining a second terminal end of the annular frame, a plurality of interconnected struts arranged in one or more rows circumferentially extending between the row of first end struts and the row of second end struts, a plurality of axial frame members including respective first ends and second ends, the first ends being connected to the row of first end struts and the second ends being connected to a first row of interconnected struts, and a plurality of support members connecting the plurality of axial frame members at connecting points that are axially located between the first ends and the second end.

Certain aspects of the disclosure also concern an assembly. The assembly can include a prosthetic device having a frame, the prosthetic device being movable between a radially expanded state and a radially compressed state, and a delivery apparatus configured to deliver the prosthetic device in the radially compressed state to a target location. The frame can include a row of outflow struts defining an outflow end of the frame, a row of inflow struts defining an inflow end of the frame, a row of interconnected struts circumferentially extending between the row of outflow struts and the row of inflow struts, a plurality of axial frame members comprising respective first ends and second ends, the first ends being connected to the row of outflow struts and the second ends being connected to the row of interconnected struts, and a plurality of support members connecting the plurality of axial frame members and extending between the row of outflow struts and the row of interconnected struts. Each outflow strut can include two angled strut portions interconnected by an apex portion. The support members can be narrower than the axial frame members, the angled strut portions of outflow struts, and the row of interconnected struts.

Certain aspects of the disclosure concern a method of assembling a prosthetic device. The method can include preparing an annular frame including a row of outflow struts defining an outflow end of the annular frame, a row of inflow struts defining an inflow end of the annular frame, a row of interconnected struts circumferentially extending between the row of outflow struts and the row of inflow struts, and a plurality of axial frame members having respective first ends and second ends, the first ends being connected to the row of outflow struts and the second ends being connected to the row of interconnected struts. The method can also include bridging the plurality of axial frame members with a plurality of support members so that the support members circumferentially extend between the row of outflow struts and the row of interconnected struts. Each outflow strut can include two angled strut portions interconnected by an apex portion. The support members can be narrower than the axial frame members, the angled strut portions of outflow struts, and the row of interconnected struts.

According to certain aspects of the disclosure, a method of assembling a prosthetic device can include preparing an annular frame including a row of outflow struts defining an outflow end of the annular frame, a row of inflow struts defining an inflow end of the annular frame, a row of interconnected struts circumferentially extending between the row of outflow struts and the row of inflow struts, a plurality of axial frame members comprising respective first ends and second ends, the first ends being connected to the row of outflow struts and the second ends being connected to the row of interconnected struts, and a plurality of support members connecting the plurality of axial frame members and extending between the row of outflow struts and the row of interconnected struts. The method can also include attaching an outflow skirt to an outer surface of the annular frame. The attaching can include connecting an outflow edge portion of the outer skirt to the plurality of support members. Each outflow strut can include two angled strut portions interconnected by an apex portion. The support members can be narrower than the axial frame members, the angled strut portions of the outflow struts, and the row of interconnected struts.

Certain aspects of the disclosure also concern a method including delivering a prosthetic device in a radially compressed state to a target location, and radially expanding the prosthetic device to a radially expanded state. The prosthetic device can include a radially expandable and compressible frame. The frame can include a row of outflow struts defining an outflow end of the frame, a row of inflow struts defining an inflow end of the frame, a row of interconnected struts circumferentially extending between the row of outflow struts and the row of inflow struts, a plurality of axial frame members comprising respective first ends and second ends, the first ends being connected to the row of outflow struts and the second ends being connected to the row of interconnected struts, and a plurality of support members connecting the plurality of axial frame members and extending between the row of outflow struts and the row of interconnected struts. Each outflow strut can include two angled strut portions interconnected by an apex portion. The support members can be narrower than the axial frame members, the angled strut portions of outflow struts, and the row of interconnected struts.

The above method can be performed on a living animal or on a simulation, such as on a cadaver, cadaver heart, anthropomorphic ghost, simulator (for example, with body parts, heart, tissue, etc. being simulated).

In certain aspects, a prosthetic valve can include an annular frame that is movable between a radially compressed configuration and a radially expanded configuration. The frame has an inflow end and an outflow end. The prosthetic valve can also include a leaflet structure positioned within the frame and configured to permit blood flow from the inflow end to the outflow end and block blood fluid flow from the outflow end to the inflow end. The frame can include a first row of angled struts defining the outflow end, a second row of angled struts upstream of the first row of angled struts, a third row of angled struts upstream of the second row of angled struts, and a plurality of axial frame members comprising respective first ends and second ends. The first ends can be connected to the second row of angled struts and the second ends can be connected to the third row of angled struts. The plurality of axial frame members can include a plurality of axially extending commissure supports and one or more axial posts located between any two immediately adjacent commissure supports. Each commissure support can be configured to support a corresponding commissure of the leaflet structure.

In certain aspects, a prosthetic valve can include an annular frame that is movable between a radially compressed configuration and a radially expanded configuration. The frame has an inflow end and an outflow end. The prosthetic valve can also include a leaflet structure positioned within the frame and configured to permit blood flow from the inflow end to the outflow end and block blood fluid flow from the outflow end to the inflow end. The frame can include a first row of angled struts defining the outflow end, a second row of angled struts upstream of the first row of angled struts, a third row of angled struts upstream of the second row of angled struts, a fourth row of angled struts upstream of the third row of angled struts, and a plurality of axial frame members comprising respective first ends and second ends. The first ends can be connected to the second row of angled struts and the second ends can be connected to the third row of angled struts. The first row of angled struts and the second row of angled struts can define a plurality of first cells of the frame. The second row of angled struts, the third row of angled struts, and the plurality of axial frame members can define a plurality of second cells of the frame. The third row of angled struts and the fourth row of angled struts can define a plurality of third cells of the frame. The number of first cells can be more than the number of second cells. The number of second cells can be equal to the number of third cells.

In certain aspects, an annular frame that is movable between a radially compressed configuration and a radially expanded configuration can include a first row of angled struts defining the outflow end, a second row of angled struts upstream of the first row of angled struts, a third row of angled struts upstream of the second row of angled struts, a fourth row of angled struts upstream of the third row of angled struts, and a plurality of axial frame members comprising respective first ends and second ends. The first ends can be connected to the second row of angled struts and the second ends can be connected to the third row of angled struts. The first row of angled struts and the second row of angled struts can define a plurality of first cells of the frame. The second row of angled struts, the third row of angled struts, and the plurality of axial frame members can define a plurality of second cells of the frame. The third row of angled struts and the fourth row of angled struts can define a plurality of third cells of the frame. The first cells can be smaller than the third cells and the third cells can be smaller than the second cells when the frame is in the radially expanded configuration.

In certain aspects, a prosthetic valve can include an annular frame that is radially expandable and compressible. The annular frame can include a row of outflow struts defining an outflow end of the frame, a row of inflow struts defining an inflow end of the frame, an intermediate row of angled struts located axially between the row of outflow struts and the row of inflow struts, a plurality of axial frame members bridging the row of outflow struts and the intermediate row of angled struts, and a plurality of support structures connecting the plurality of axial frame members and the intermediate row of angled struts. Each support structure can include at least two angled support arms connecting two immediately adjacent axial frame members and at least one axial support member bridging the at least two angled support arms and the intermediate row of angle struts.

In certain aspects, a prosthetic valve can include an annular frame that is radially expandable and compressible. The annular frame can include a row of outflow struts defining an outflow end of the frame, a row of inflow struts defining an inflow end of the frame, first and second intermediate rows of angled struts located axially between the row of outflow struts and the row of inflow struts, a plurality of axial frame members bridging the row of outflow struts and the first intermediate row of angled struts, and a plurality of support structures connecting the plurality of axial frame members and the first and second intermediate rows of angled struts. The second intermediate row of angled struts are upstream of the first intermediate row of angled struts. Each support structure can include at least two angled support arms connecting two immediately adjacent axial frame members and two or more axial support members bridging the at least two angled support arms and the first and second intermediate rows of angle struts.

In some examples, a prosthetic valve or a frame of a prosthetic valve can comprise one or more of the components recited in Examples 1-95 and 107-187 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a prosthetic valve, according to one example.

FIG. 2 is a perspective view of a prosthetic valve, according to another example.

FIG. 3 is a perspective view of a prosthetic valve, according to yet another example.

FIG. 4A is a perspective view of an annular frame of a prosthetic valve, according to one example, wherein the annular frame is in an expanded configuration.

FIG. 4B is a flattened view of the expanded frame of FIG. 4A.

FIG. 4C is a flattened view of a portion of the frame of FIG. 4A, wherein the frame is in a compressed configuration.

FIG. 4D is a flattened view of a portion of the expanded frame of FIG. 4A, according to one example.

FIG. 5A is a perspective view of an annular frame of a prosthetic valve, according to another example, wherein the annular frame is in an expanded configuration.

FIG. 5B is a flattened view of the expanded frame of FIG. 5A.

FIG. 5C is a flattened view of a portion of the frame of FIG. 5A, wherein the frame is in a compressed configuration.

FIG. 6A depicts an outflow strut of a frame comprising two angled strut portions connected by a U-shaped bend, according to one example.

FIG. 6B depicts an inflow strut of a frame forming comprising two angled strut portions connected by a U-shaped bend, according to one example.

FIG. 7A depicts one column of a frame, according to one example.

FIG. 7B depicts one column of a frame, according to another example.

FIG. 7C depicts one column of a frame, according to yet another example.

FIG. 8A depicts a flattened view of a portion of an annular frame in the expanded configuration, according to another example.

FIG. 8B depicts a flattened view of a portion of an annular frame in the expanded configuration, according to another example.

FIG. 9A depicts a flattened view of a portion of an annular frame in the expanded configuration, according to another example.

FIG. 9B depicts a flattened view of a portion of an annular frame in the expanded configuration, according to another example.

FIG. 10 is a perspective view of a frame of a prosthetic valve, according to another example.

FIG. 10A is a flattened view of a portion of the frame of FIG. 10, the frame being in an expanded configuration.

FIG. 10B is a flattened view of a portion of the frame of FIG. 10, the frame being in a compressed configuration.

FIG. 11 is a flattened view of a portion of a frame for a prosthetic valve, according to another example.

FIG. 12 is a flattened view of a portion of a frame for a prosthetic valve, according to another example.

FIG. 13 is a flattened view of a portion of a frame for a prosthetic valve, according to another example.

FIG. 14 is a flattened view of a portion of a frame for a prosthetic valve, according to another example.

FIG. 15 is a flattened view of a portion of a frame for a prosthetic valve, according to another example.

FIG. 16 is a flattened view of a portion of a frame for a prosthetic valve, according to another example.

FIG. 17 is a flattened view of a portion of a frame for a prosthetic valve, according to another example.

FIG. 18 is a flattened view of a portion of a frame for a prosthetic valve, according to another example.

FIG. 19 is a flattened view of a portion of a frame for a prosthetic valve, according to another example.

FIG. 20 is a side view of a delivery apparatus configured to deliver and implant a prosthetic valve at a target implantation site, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. Further, as used herein, “and/or” means “and” or “or,” as well as “and” and “or.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### General Overview of Prosthetic Valves

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

According to certain examples, any of the prosthetic valves disclosed herein can be adapted to be implanted in the native aortic annulus. In other examples, any of the prosthetic valves disclosed herein can be adapted to be implanted in other native annuluses of the heart (for example, the pulmonary, mitral, and tricuspid valves). In certain examples, the disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. Additionally, the disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

### Exemplary Components of Prosthetic Valves

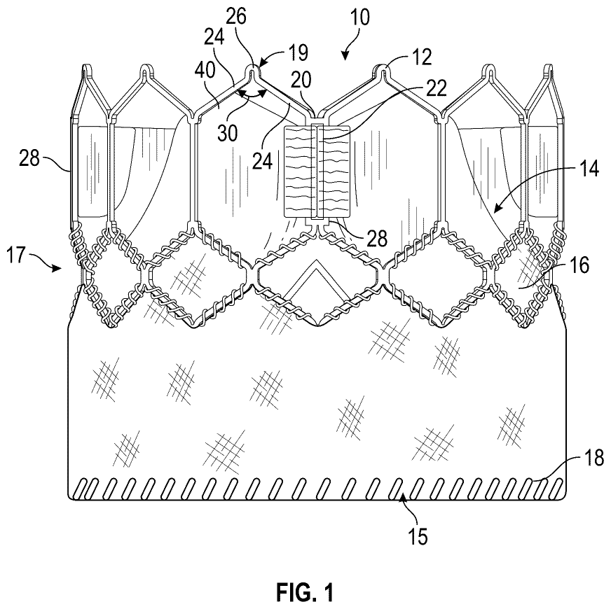

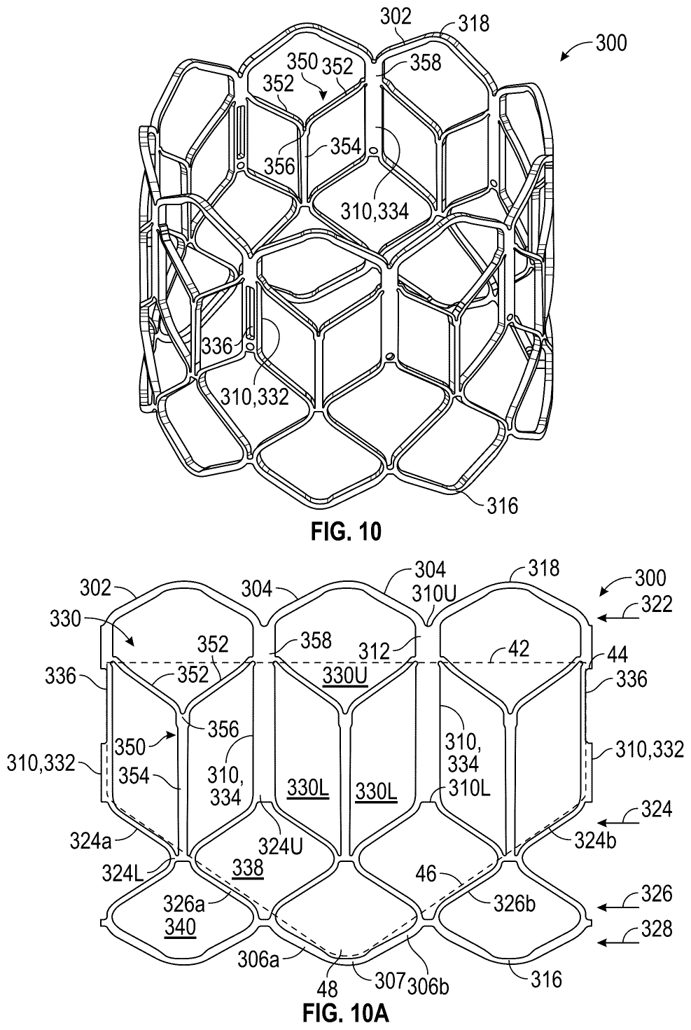

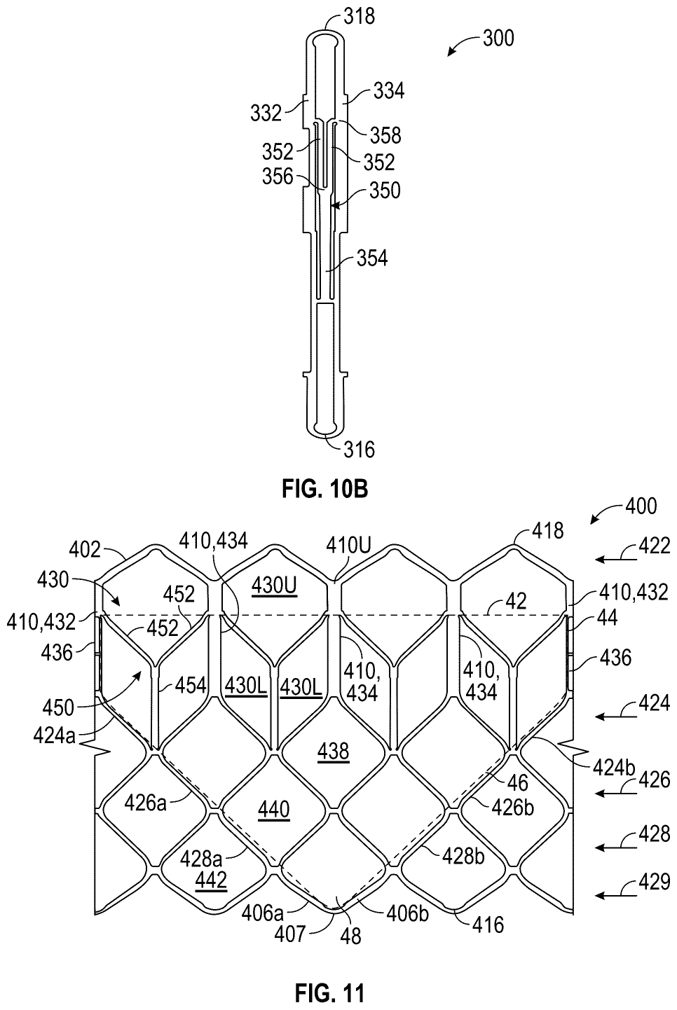

FIG. 1 illustrates an exemplary prosthetic valve 10 that can be advanced through a patient's vasculature, such as to a native heart valve, by a delivery apparatus, such as the exemplary delivery apparatus shown in FIG. 10.

As shown, the prosthetic valve 10 comprises a stent or frame 12, a valvular structure 14, an inner skirt 16, and a perivalvular outer sealing member or outer skirt 18. The frame 12 can be similar to, or replaced by, any one of the frames described herein.

The frame 12 can have an inflow end 15, an outflow end 19, and an intermediate portion 17 between the inflow end 15 and the outflow end 19. The valvular structure 14 can be configured to allow unidirectional flow of liquid through the prosthetic valve 10. In certain examples, the valvular structure 14 can include a plurality of leaflets 40, collectively forming a leaflet structure, which is configured to permit blood flow from the inflow end 15 to the outflow end 19 and block blood fluid flow from the outflow end 19 to the inflow end 15.

For example, the valvular structure 14 can comprise three leaflets 40, which can be arranged to collapse in a tricuspid arrangement. In other examples, there can be greater or fewer number of leaflets (for example, one leaflet, two leaflets, or more than three leaflets). The leaflets 40 can be secured to one another at their adjacent sides to form commissures 22 (or “commissure tabs”) of the leaflet structure 14. The lower (or inflow) edge of valvular structure 14 can define an undulating, curved scalloped shape, and thus can be referred to as a “scallop line.” In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

Additional examples of leaflet assemblies and methods for attaching leaflets to a frame are described in Provisional U.S. Patent Application Nos. 63/300,302, filed Jan. 18, 2022, and 63/278,922, filed Nov. 12, 2021, which are incorporated by reference herein.

The frame 12 can be radially compressible (collapsible) and expandable (for example, the radially expanded configuration is shown in FIG. 1). The frame 12 can comprise a plurality of interconnected angled struts 24. A plurality of apices 26 that are spaced circumferentially apart can be formed at the inflow end 15 and the outflow end 19 of the frame 12. In FIG, 1, only the apices 26 at the outflow end 19 are visible, whereas the apices 26 at the inflow end 15 are covered by the outer skirt 18. Each apex 26 can be formed at a junction between two angled struts 24 at either the inflow end 15 or the outflow end 19. FIG. 1 depicts one example frame design where each apex 26 forms a U-shaped bend between the two angled struts 24. In some examples, an angle 30 between the two angled struts 24, connected at the apex 26, can be in a range of 90 to 120 degrees when the frame 12 is in the radially expanded configuration.

The frame 12 can be formed with a plurality of circumferentially spaced commissure windows 20 that are adapted to mount the commissures 22 of the valvular structure 14 to the frame 12. For example, the frame 12 can include a plurality of axial frame members 28 bridging two adjacent rows of the angled struts 24 that are closest to the outflow end 19. The commissure windows 20 can be formed as through-thickness openings (for example, axially extending slots) on selected axial frame members 28. The commissures 22 can be inserted through the commissure windows 20 and secured to the selected axial frame members 28. Thus, the selected axial frame members 28 having the commissure windows 20 can also be referred to as commissure supports.

The inner skirt 16 (and any inner skirts described herein) and outer skirt 18 (and any other outer skirts described herein) can be formed from any of various suitable biocompatible materials, including any of various synthetic materials, including fabrics (for example, polyethylene terephthalate fabric) or natural tissue (for example, pericardial tissue). The inner skirt 16 can be arranged on and/or coupled to an inner surface of the frame 12, while the outer skirt 18 can be arranged on and/or coupled to an outer surface of the frame 12. The outer skirt 18 can function as a scaling member for the prosthetic valve 10 by sealing against the tissue of the native valve annulus and helping to reduce paravalvular leakage past the prosthetic valve 10. The inner skirt 16 can function as a sealing member to prevent or decrease perivalvular leakage, to anchor the leaflets 40 to the frame 12, and/or to protect the leaflets 40 against damage caused by contact with the frame 12 during crimping and during working cycles of the prosthetic valve 10. In some examples, the inflow edge of the leaflets 40 can be sutured to the inner skirt 16 generally along the scallop line. The inner skirt 16 can in turn be sutured to adjacent angled struts 24 of the frame 12. In other examples, the leaflets 40 can be sutured directly to the frame 12 along the scallop line via stitches.

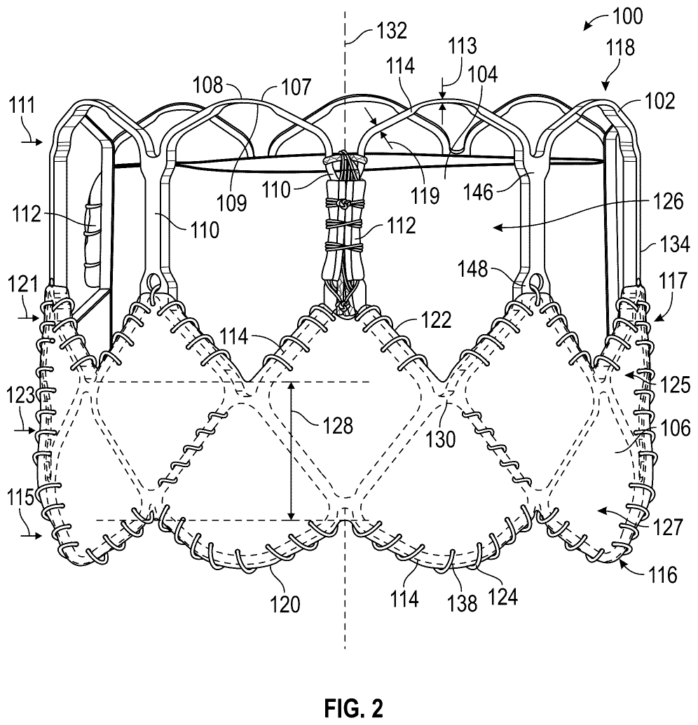

FIG. 2 depicts another exemplary prosthetic valve 100 comprising a radially expandable and/or compressible annular frame 102, a valvular structure comprising a plurality of leaflets 104 mounted within the frame 102, and an outer skirt 106 secured to and around an outer surface 134 of the frame 102. The frame 102 can be similar to, or replaced by, any one of the frames described herein.

The frame 102 can comprises a plurality of angled struts 114 and a plurality of apices (for example, outflow apices 108 and inflow apices 138) that are spaced circumferentially apart around an outflow end 118 and an inflow end 116 of the frame 102. Each apex 108 or 138 is formed at a junction between two angled struts 114 at either the outflow end 118 or the inflow end 116.

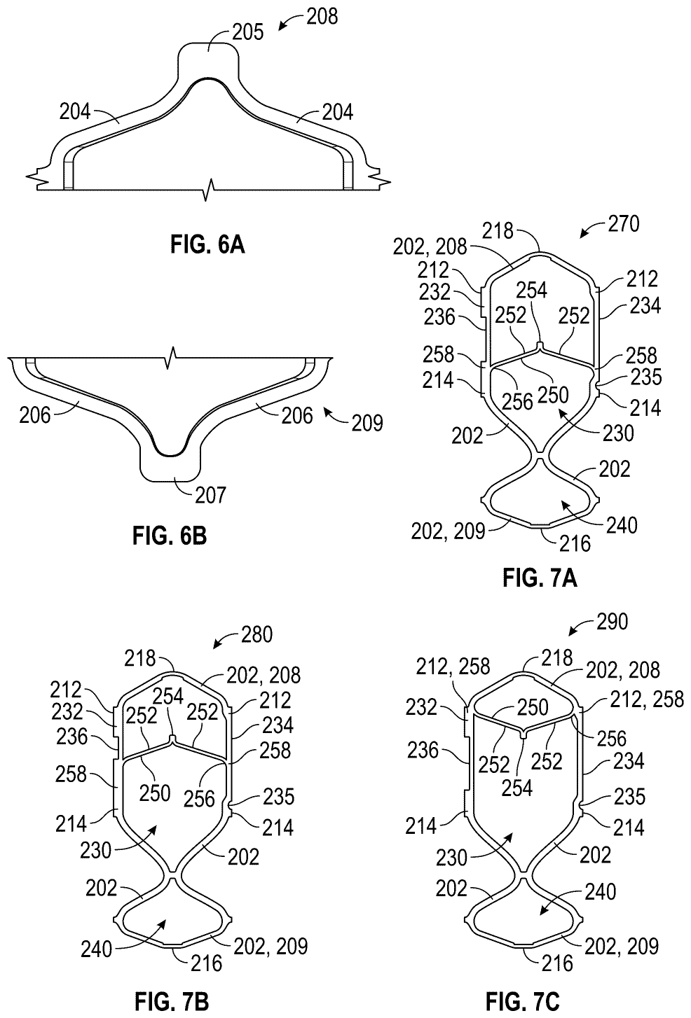

Each apex 108 or 138 can have a more rounded or flattened (for example, less pointed) shape as compared to the U-shaped apices 26 of FIG. 1. For example, each apex 108 (or 138) can include a curved or relatively flat outer surface 107 and an arcuate or curved inner depression 109 disposed opposite the outer surface 107 (FIG. 6). The inner depression 109 can form a thinned region at the apex 108 (or 138), having a width 113 that is smaller or narrower than a width 119 of the angled struts 114. This thinned region of the apex 108 (or 138) can spread stresses experienced by the frame 102 away from the apex and across both the angled struts 114 extending from either side of the apex 108 (or 138).

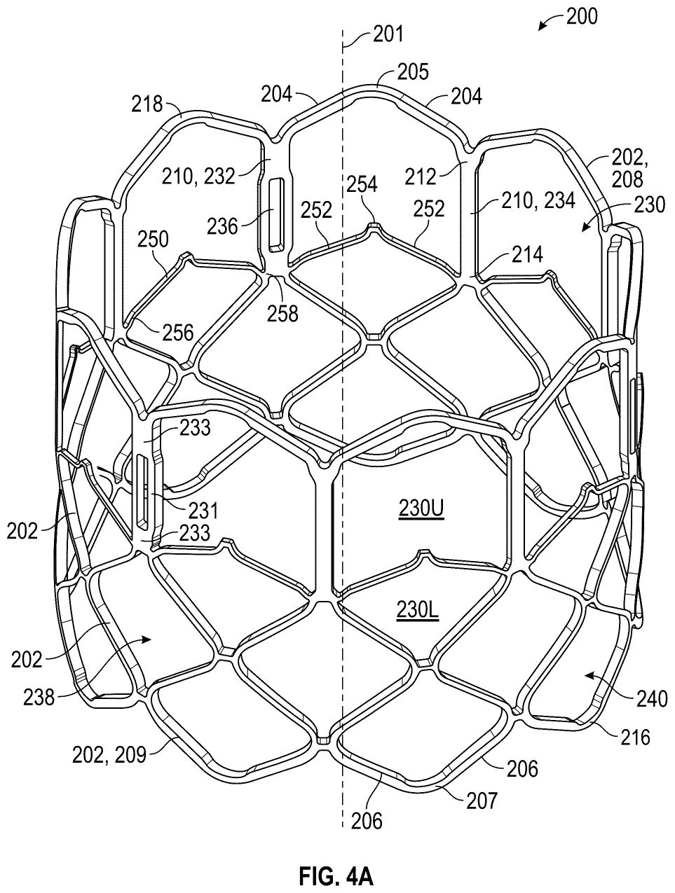

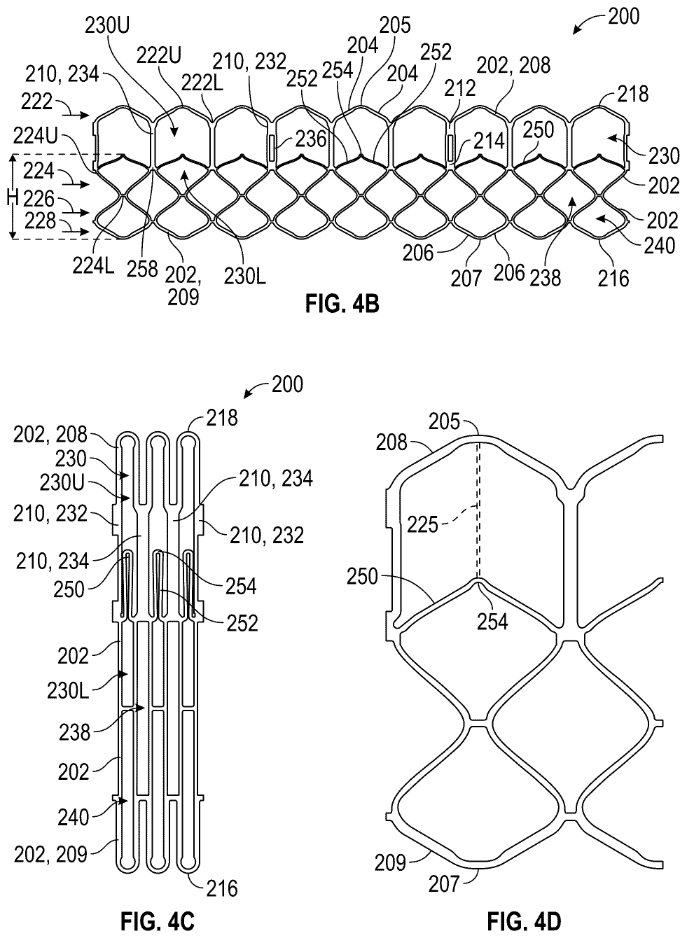

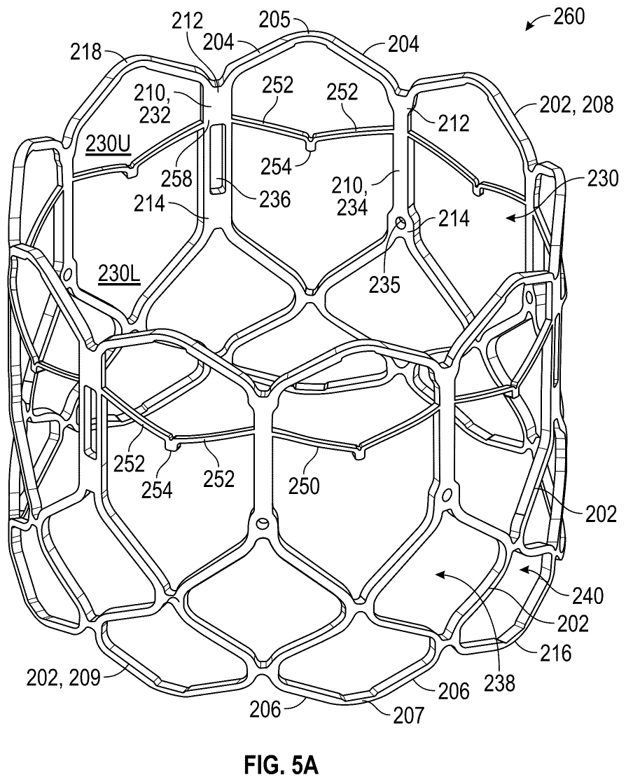

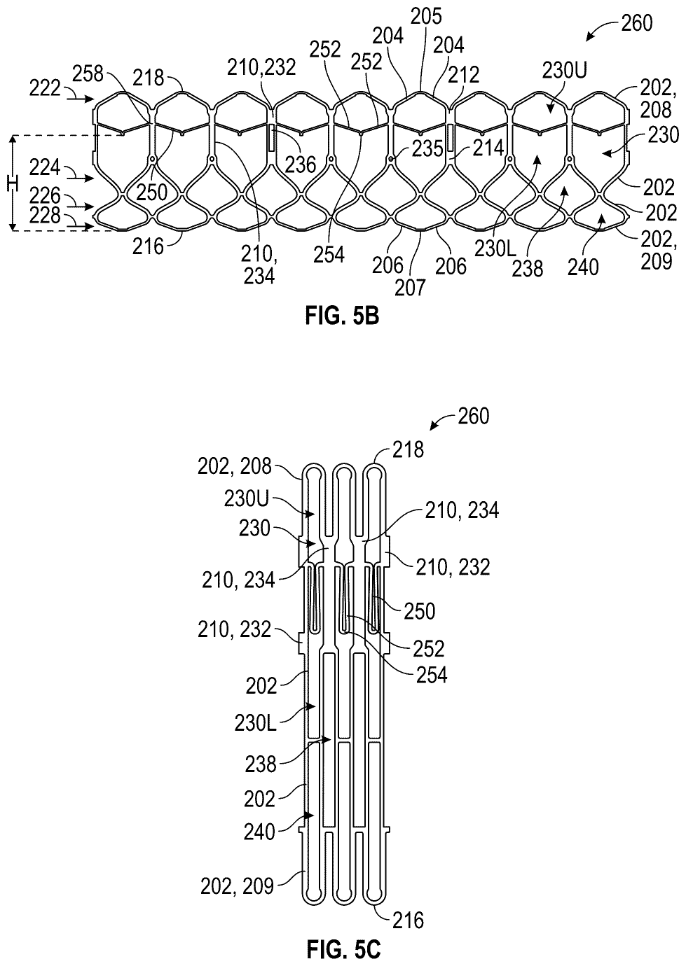

As described herein, the “width” of a frame component (for example, an angled strut or strut portion, an apex or apex portion, an axial frame member, etc.) is measured between opposing locations on opposing surfaces of such frame component that extend between the radially facing inner and outer surfaces of the frame component. The width of the frame component can be measured, for example, by flattening the frame on a planar surface (see, for example, FIGS. 4B and 5B) and measuring a cross-section (that is, transverse section) width of the frame component taken along a short axis that is perpendicular to the longitudinal axis of the frame component and parallel to the plane of the page.

The angled struts 114 can be arranged in multiple rows between the inflow end 116 and the outflow end 118. For example, FIG. 2 shows four rows of angled struts 114: a first row 111 of angled struts defining the outflow end 118, a second row 121 of angled struts upstream of the first row 111 of angled struts, a third row 123 of angled struts upstream of the second row 121 of angled struts, and a fourth row 115 of angled struts defining the inflow end 116. The first row 111 of angled struts can also be referred to as outflow struts, and the fourth row 115 of angled struts can also be referred to as inflow struts. In other examples, the frame 102 can include different number of rows (for example, 3, 5, or more) of angled struts. As described herein, a frame component (for example, a row of angled struts) is deemed to be upstream of a reference object (for example, another row of angled struts) if the frame component is closer to the inflow end 116 (or farther away from the outflow end 118) than the reference object.

The frame 102 can comprise a plurality of axially extending struts, also referred to herein as axial frame members 110, some of which can define commissure windows (similar to 20) therein. Lateral portions of adjacent leaflets 104 can be paired together and extend through the commissure windows, thereby forming commissures 112 secured to the frame 102.

The axial frame members 110 can bridge the first row 111 and the second row 121 of the angled struts. For example, upper ends 146 of the axial frame members 110 can be connected to lower ends of the first row 111 of angled struts, and lower ends 148 of the axial frame members 110 can be connected to upper ends of the second row 121 of angled struts.

The axial frame members 110, together with the first row 111 of angled struts and the second row 121 of angled struts, can form a circumferentially extending row of open outflow cells 126 (which can also be referred to as “first cells” or “upper cells”). The first row 111 of angled struts can form the upper or outflow edges of the outflow cells, and the second row 121 of angled struts can form the lower or inflow edges of the outflow cells 126. The rows of angled struts upstream of the first row 111 (for example, rows 121, 123, 115) can be interconnected so as to form additional rows of open cells. For example, FIG. 2 shows a second row of intermediate cells 125 disposed proximate an intermediate portion 117 of the frame 102 and a third row of inflow cells 127 disposed at the inflow end 116 of the frame 102. The outflow cells 126 can have a hexagonal shape when the frame 102 is radially expanded. In certain examples, the outflow cells 126 can be more axially elongated and have a larger open area than the intermediate cells 125 and the inflow cells 127.

The outer skirt 106 of the prosthetic valve 100 can include an inflow edge portion 120 (or lower edge portion) that is secured to the inflow struts (for example, the fourth row 115 of angled struts) via one or more fasteners, such as whip stitches 124. The outer skirt 106 can also include an outflow edge portion 122 (or upper edge portion) that is secured (for example, via sutures 130) to the second row 121 of angled struts that form the lower or inflow edges of the outflow cells 126. In some examples, the lower ends 148 of the axial frame members 110 can include apertures 132, through which the sutures 130 can extend so as to fasten the outflow edge portion 122 of the outer skirt 106 to the lower ends 148 of the axial frame members 110.

In some instances, as shown in FIG. 2, the outflow edge portion 122 of the outer skirt 106 can extend along and follow a shape of the angled struts 114 forming the lower or inflow edges of the outflow cells 126, thereby causing the outflow edge portion 122 to affix to (for example, via the sutures 130) follow a zig-zag pattern of the second row 121 of angled struts. While such a configuration can result in a tight attachment of the outflow edge portion 122 of the outer skirt 106 to the frame 102, it can also result in the outer skirt 106 having a relatively short minor axial height 128, which is the height measured along portions of the outer skirt 106 that extend axially from the non-apical junctions near the inflow end 116 (for example, the junctions where the third row 123 and the fourth row 115 of angled struts are connected) to the bottom junctions of the outflow cells 126, as illustrated in FIG. 2.

In certain circumstances, it may be desirable to extend the outer skirt beyond the lower or inflow edges of the outflow cells 126, for example, above (or closer to the outflow end 118 than) the second row 121 of angled struts. For example, it may be desirable to extend the outflow edge portion 122 of the outer skirt 106 toward the outflow end 118 (for example, closer to a mid-height of the outflow cells 126) in order to increase a surface area for improved sealing to reduce paravalvular leakage at the implantation site.

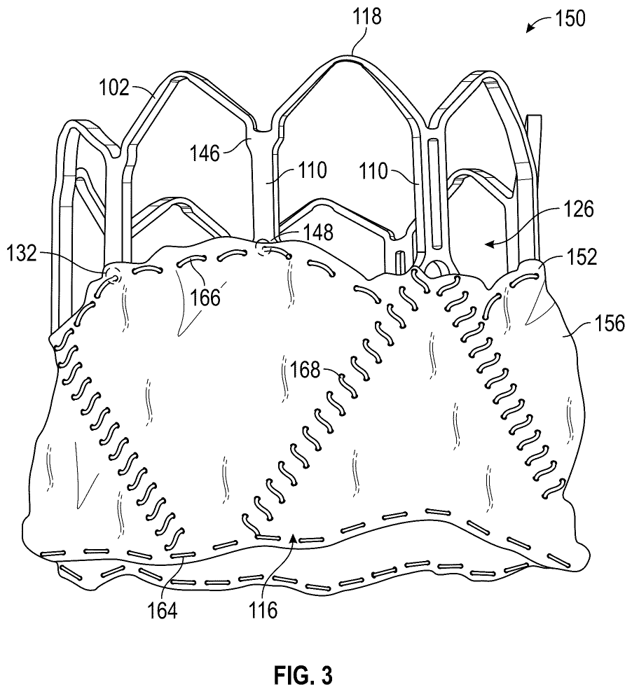

FIG. 3 is a perspective view of another prosthetic valve 150 comprising the frame 102 and an outer skirt 156 secured to the frame 102. The frame 102 can be similar to, or replaced by, any one of the frames described herein. The prosthetic valve 150 can include leaflets 104 assembled to the frame 102, as shown in FIG. 2 and described above.

As shown in FIG. 3, the outer skirt 156 can extend around an outer surface of the frame 102, from an inflow end 116 (which is covered by the outer skirt 156) toward an outflow end 118 of the frame 102. In some examples, the outer skirt 156 can be secured to struts of the frame 102 at the inflow end 116 by one or more sutures 164.

In some examples, an outflow edge portion 152 of the outer skirt 156 can be secured to the lower ends 148 of the axial frame members 110 and/or to upper ends of the second row 121 of angled struts by one or more sutures 166. In some examples, a portion of the sutures 166 can extend through and be secured to the apertures 132 at the lower ends 148 of the axial frame members 110 (apertures 132 shown in dashed lines in FIG. 3 to denote their position underneath the outer skirt 156).

In some examples, the sutures 166 and/or the sutures 164 can be in-and-out stitches. In some examples, the sutures 166 can be continuous between adjacent apertures 132 (for example, extending in and out of the outer skirt 156). In other examples, the sutures 166 can be separated pieces that individually connect the outflow edge portion 152 of the outer skirt 156 to respective axial frame members 110.

In some examples, as shown in FIG. 3, additional sutures 168, which can be configured as whip stitches, can further secure the outer skirt 156 to angled struts of the frame 102 disposed and extending between the inflow end 116 and the lower ends 148 of selected axial frame members 110 (for example, the axial frame members having commissure windows).

Unlike the prosthetic valve 100 of FIG. 2 where the outflow edge portion 122 of the outer skirt 106 follows the zig-zag pattern of the second row 121 of angled struts, the prosthetic valve 150 of FIG. 3 can have a larger outer skirt 156 whose outflow edge portion 152 can have about the same height (that is, having substantially constant axial distance relative to the inflow end 116) around the circumference of the frame 102. As such, the outer skirt 156 can cover at least a lower portion of the outflow cells 126.

To provide further structural support of the outer skirt 156, for example, to reduce the likelihood that the outflow edge portion 152 of the outer skirt 156 will extend through or loosely dangle across the outflow cells 126 (for example, due to the relatively large size of the outflow cells 126), rungs of support members extending between the axial frame members 110 can be added to the frame 102 so that the outflow edge portion 152 of the outer skirt 156 can be further attached to the support members. Examples of frames having runs of support members are described further below.

Further details regarding the prosthetic valves and various components of prosthetic valves, including the related delivery apparatus/catheters/systems are described in WIPO Patent Application Publication No. WO 2018/222799, which is incorporated herein by reference. Additional examples of the frame are further described in U.S. Provisional Patent Application No. 63/401,538, filed Aug. 26, 2022, which is also incorporated herein by reference.

### Exemplary Frames Having Rungs of Support Members

FIGS. 4A-4D depict a frame 200 which can be incorporated in any of the prosthetic valves (for example, 10, 100, 150) described herein. Similarly, the frame 200 can be moved between a radially compressed configuration and a radially expanded configuration.

As shown in FIG. 4A, the frame 200 has an inflow end 216 (also referred to as a “second terminal end” or “inflow terminal end”), an outflow end 218 (also referred to as a “first terminal end” or “outflow terminal end”), and a plurality of angled struts 202 arranged in a plurality of rows circumferentially extending between the inflow end 216 and the outflow end 218.

As depicted in FIG. 4B, the frame 200 has four rows of angled struts 202: a first row 222 of angled struts defining the outflow end 218, a second row 224 of angled struts upstream of the first row 222, a third row 226 of angled struts upstream of the second row 224, and a fourth row 228 of angled struts upstream of the third row 226 and defining the inflow end 216. The first row 222 of angled struts can also be referred to as “outflow struts” 208 or “first end struts,” and the fourth row 228 of angled struts can also be referred to as “inflow struts” 209 or “second end struts.” Although four rows of angled struts are depicted in FIGS. 4A-4D, it should be understood that in other examples, the frame 200 can have fewer (for example, three) or more (for example, five, six, seven, etc.) rows of angled struts 202 between the inflow end 116 and outflow end 218.

In the depicted example, each outflow strut 208 can include two angled strut portions 204 interconnected by an apex portion 205. For example, each apex portion 205 can curve between a pair of two angled strut portions 204 of a corresponding outflow strut 208. In certain examples, each apex portion 205 can have an arc length that extends along at least 25% of a total arc length of the corresponding outflow strut 208. Similarly, each inflow strut 209 can include a pair of angled strut portions 206 interconnected by a curved apex portion 207 (see, for example, FIGS. 4B and 4D). In certain examples, each apex portion 207 can have an arc length that extends along at least 25% of a total arc length of the corresponding inflow strut 209. Similar to apices 108 and 138 of FIG. 2, the apex portions 205, 207 can be thinner or narrower (for example, have a smaller width) than the corresponding angled strut portions 204, 206.

Similar to apices 108 and 138 of FIG. 2, the apex portions 205, 207 are more rounded and flattened. In other examples, as depicted in FIG. 6A, the apex portion 205 can form a U-shaped bend (similar to apices 26 of FIG. 1) between two angled strut portions 204 of a corresponding outflow strut 208. Similarly, as depicted in FIG. 6B, the apex portion 207 can form a U-shaped bend between two angled strut portions 206 of a corresponding inflow strut 209.

As depicted in FIGS. 4A-4D, the frame 200 also includes a plurality of axially extending posts or axial frame members 210 bridging two rows of angled struts 202 (for example, the first row 222 and the second row 224) that are closest to the outflow end 218. The axial direction of the frame 200 is indicated by a longitudinal axis 201, which extends from the inflow end 216 to the outflow end 218.

Each axial frame member 210 can have a first end 212 connected to the first row 222 of angled struts and a second end 214 connected to the second row 224 of angled struts. For example, as depicted in FIG. 4B, the first row 222 of angled struts can be joined together to form a first set of alternating upper ends (or peaks) 222U at the outflow apex portions 205 and lower ends (or valleys) 222L between adjacent lower ends of adjacent strut portions 204. The upper ends 222U can define the outflow end 218 and the lower ends 222L are closer to the inflow end 216 than the upper ends 222U. The second row 224 of angled struts can be joined together to form a second set of alternating upper ends (or peaks) 224U and lower ends (or valleys) 224L. The upper ends 224U are closer to the outflow end 218 than the lower ends 224L. As shown, the axial frame members 210 can respectively connect the lower ends 222L of the first row 222 of angled struts to the upper ends 224U of the second row 224 of angled struts.

According to certain examples, selected axial frame members 210 can be configured as commissure supports 232, each of which can support a corresponding commissure of a valvular structure (for example, comprising leaflets similar to 40 or 104, as described above) mounted within the frame 200. In the depicted example, the frame 200 includes three axially extending commissure supports 232 configured to respectively receive three commissures formed by three leaflets. In the illustrated example, each commissure support 232 can include an opening or commissure window 236 which has a substantially rectangular shape that is shaped and sized to receive a commissure of two adjacent leaflets therethrough. In other examples, the commissure window 236 can have any of various shapes (for example, square, oval, square-oval, triangular, L-shaped, T-shaped, C-shaped, etc.).

The commissure supports 232 (and thus the commissure windows 236) can be angularly spaced apart from one another around the circumference of frame 200. The spacing between the commissure supports 232 may or may not be even.

In some examples, each commissure window 236 can be axially offset from the first end 212 and second end 214 of the corresponding commissure support 232. As an example, the axial position of the commissure windows 236 can be selected such that when the leaflets are attached to the frame 200 via the commissure windows 236, the free edge portions (for example, outflow edge portions) of the leaflets will not protrude from or past the outflow end 218 of the frame 200.

Other axial frame members 210 (that is, not commissure support 232), which can also be referred to as axial posts 234, are located between any two immediately adjacent commissure supports 232. For example, the frame 200 can have two axial posts 234 located between each pair of immediately adjacent commissure supports 232.

Although the frame 200 depicted in FIGS. 4A-4B has a total of nine axial frame members 210 (including three commissure supports 232 and six axial posts 234), it is to be understood that the frame 200 can have more or less than nine axial frame members 210 (for example, with more or less than three commissure supports 232 and/or different number of axial posts 234).

The plurality of rows of angled struts 202 and the axial frame members 210 can form a plurality of rows of open cells of the frame 200. For example, the first row 222 of angled struts, the second row 224 of angled struts, and the plurality of axial frame members 210 can define a plurality of first cells 230 (also referred to as “outflow cells”) of the frame 200. The first cells 230 can have a hexagonal shape when the frame 200 is in the expanded configuration.

In certain examples, the second row 224 of angled struts and the third row 226 of angled struts can define a plurality of second cells 238 of the frame 200. Likewise, the third row 226 of angled struts and the fourth row 228 of angled struts define a plurality of third cells 240 (also referred to as “inflow cells”) of the frame 200. Each of the second cells 238 and the third cells 240 can have a quadrilateral shape when the frame 200 is in the expanded configuration.

Generally, the first cells 230 can have a larger axial length (and a larger area) than the second cells 238 and the third cells 240, resulting in more open space or larger openings at near the outflow end 218 for blood flow and coronary access.

As described herein, the plurality of rows of open cells 230, 238, and 240 can be arranged in a plurality of columns of the frame 200 defined by the axial frame members 210. For example, the frame 200 depicted in FIG. 4B has nine columns delineated by the nine axial frame members 210, where each column is formed by one first cell 230, one third cell 240 adjacent to the first cell 230, and two partial second cells 238 located between the first cell 230 and the third cell 240.

In certain examples, the second cells 238 and the third cells 240 have about the same size when the frame 200 is in the expanded configuration. In other examples, the size and/or shape of the second cells 238 and third cells 240 can differ when the frame 200 is in the expanded configuration. For example, when the frame 200 is in the expanded configuration, the second cells 238 can approximate a diamond shape (for example, the angled struts in the second row 224 and the third row 226 defining each second cell 238 can have equal length), wherein the third cells 240 can have a non-diamond shape, for example, the angle formed at each apex portion 207 (that is, the angle between a pair of angled strut portions 206) can be larger than the opposing angle formed between a pair of angled struts in the third row 226.

As depicted in FIGS. 4A-4D, the frame 200 can further include rungs of support members 250 connecting the plurality of axial frame members 210 and extending between the first row 222 of angled struts and the second row 224 of angled struts. Some of the support members 250 can connect between pairs of immediately adjacent axial posts 234. Some of the support members 250 can connect commissure supports 232 to immediately adjacent axial posts 234. As described herein, the support members 250 can be used for attachment of an outer skirt (similar to 18, 106, 156) mounted on the outer surface of the frame 200. For example, an outflow edge portion (for example, 122, 152) of the outer skirt can be connected to the support members 250 via sutures.

The support members 250 can be configured to be axially foldable. For example, each support member 250 can include two arm portions 252 (also referred to as “angled support arms” hereinafter) and a joint portion 254 connecting the two arm portions 252 (for example, in the form of angled struts), and the support member 250 can fold at the joint portion 254 when the frame 200 moves from the radially expanded configuration (see, for example, FIG. 4A) to the radially compressed configuration (see, for example, FIG. 4C). When folded, the two arm portions 252 of each support member 250 can be configured to extend substantially parallel to and between two immediately adjacent axial frame members 210.

In the example depicted in FIGS. 4A-4B, the joint portion 254 of each support member 250 forms a U-shaped bend between the corresponding pair of arm portions 252 and extends upward (that is, toward the outflow end 218). In another example as depicted in FIG. 4D, the joint portion 254 of each support member 250 can curve between the corresponding pair of arm portions 252 (similar to the more rounded or flat apex portions 205, 207). In some examples, the two arm portions 252 can form an obtuse angle (for example, >90 degrees) at the joint portion 254 when the frame 200 is radially expanded.

Each support member 250 can connect two immediately adjacent axial frame members 210. In some examples, the two arm portions 252 of each support member 250 have the same or substantially the same length and are configured to be symmetric about the corresponding joint portion 254. In the example depicted in FIGS. 4A-4D, each arm portion 252 is substantially straight, except for an end portion 256 opposite to the joint portion 254, which can be curved. The end portions 256 are where the support members 250 connect to the corresponding axial frame members 210. Thus, the end portions 256 can also be referred to as attachment portions.

The support members 250 can be configured to have a smaller width than any other strut components of the frame 200. For example, the support members 250 can be narrower than the axial frame members 210 (including the commissure supports 232) and the angled struts 202 in any one of the rows (for example, 222, 224, 226, 228). As described herein, when an outflow strut 208 or an inflow strut 209 has a thinned apex portions (for example, 205, 207), the width of the outflow strut 208 or the inflow strut 209 refers to the width of the corresponding angled strut portions 204, 206. Thus, as described herein, the support members 250 are narrower than the angled strut portions 204 of the outflow struts 208 and the angled strut portions 206 of the inflow struts 209.

In certain examples, the angled struts 202 in any one of the rows (for example, 222, 224, 226, 228) are narrower than the axial frame members 210. The larger width of the axial frame members 210 allows a larger contact area when the leaflets contact the wider inner surfaces of the axial frame members 210 during systole, thereby distributing the stress and reducing the extent to which the leaflets may fold over the axial frame members 210, radially outward through the first cells 230. As a result, a long-term durability of the leaflets can be increased.

Increasing the width of the axial frame members 210 can reduce the area of first cells 230, particularly when the frame 200 is radially compressed. As described herein, the support members 250 are configured to be sufficiently thin or narrow so that the folded support members 250 can fit within the narrowed (and elongated) first cells 230 when the frame 200 is radially compressed (see, for example, FIG. 4C). This can be achieved, for example, by forming the support members 250 from a fully annealed metal, such as cobalt-chromium, stainless steel, or the like.

In certain examples, the angled struts in the first row 222 (that is, outflow struts 208) and the fourth row 228 (that is, inflow struts 209) can have about the same width. The angled struts in the second row 224 and the third row 226 can have about the same width.

In certain examples, the angled struts in the second row 224 and the third row 226 are narrower than the angled struts in the first row 222 (that is, outflow struts 208) and the fourth row 228 (that is, inflow struts 209).

In certain examples, a width of the support members 250 can range between 0.1 mm and 0.3 mm, or between 0.18 mm and 0.22 mm, inclusive. In one specific example, the width of the support member 250 is about 0.20 mm.

In certain examples, a width of the angled strut portions 204, 206 of the outflow struts 208 and inflow struts 209 can range between 0.3 mm and 0.5 mm, or between 0.38 mm and 0.42 mm, inclusive. In one specific example, the width of the angled strut portions 204, 206 is about 0.4 mm.

In certain examples, a width of the apex portions 205, 207 of the outflow struts 208 and inflow struts 209 can range between 0.15 mm and 0.25 mm, or between 0.18 mm and 0.22 mm, inclusive. In one specific example, the width of the apex portions 205, 207 is about 0.20 mm.

In certain examples, a width of any angled struts 202 other than the outflow struts 208 and inflow struts 209 (for example, the angled struts in the second row 224 and the third row 226) can range between 0.15 mm and 0.35 mm, or between 0.23 mm and 0.27 mm, inclusive. In one specific example, the width of any angled struts 202 in the second row 224 and the third row 226 is about 0.25 mm.

In certain examples, a width of the axial frame members 210 can range between 0.2 mm and 2 mm, or between 0.8 mm and 1.2 mm, inclusive. In certain examples, the commissure supports 232 (that is, the axial frame members having commissure windows 236) are wider than axial posts 234. In any examples described herein, if the commissure support 232 has a variable width along its axial length (for example, the portion of the commissure support 232 surrounding the commissure window 236 can be wider than portions of the commissure support that is above and/or below the commissure window), the width of the commissure support 232 refers to the largest width along the entire axial length of the commissure support 232. In some examples, the axial posts 234 can have a width ranging between 0.3 mm and 1.5 mm, or between 0.6 mm and 1 mm, inclusive. In certain examples, each axial post 234 can have a substantially uniform width between the first end 212 and second end 214 of the axial post 234. In some examples, each commissure support 232 can have a nonuniform width between the first end 212 and second end 214 of the commissure support 232. For example, each commissure support 232 can have a middle portion 231 defining the commissure windows 236 and two end portions 233 located above and below the commissure windows 236, and the middle portion 231 can be wider than the two end portions 231. In one particular example, the middle portion 231 of the commissure support 232 can have a width ranging between 1.2 mm and 1.6 mm, inclusive (for example, 1.4 mm). The end portions 233 located above and below the commissure windows 236 can have a width ranging between 1.0 and 1.4 mm, inclusive (for example, 1.2 mm). In certain examples, the commissure window 236 can have a width ranging between 0.5 mm and 0.7 mm, inclusive (for example, 0.6 mm).

As described herein, each support member 250 can be connected to two immediately adjacent axial frame members 210 at respective connecting points 258, which can be the first ends 212, the second ends 214, or any locations between the first ends 212 and second ends 214 of the axial frame members 210.

In the example depicted in FIGS. 4A-4D, the connecting points 258 are at the second ends 214 of the axial frame members 210. The support members 250 can divide the first cells 230 into respective upper cell portions 230U and lower cell portions 230L, the upper cell portions 230U being closer to the outflow end 218 than the lower cell portions 230L. In the depicted example, the upper cell portions 230U are larger than the lower cell portions 230L when the frame 200 is in the radially expanded configuration. In other examples, the lower cell portions 230L can be larger than or have about the same size as the upper cell portions 230U when the frame 200 is in the radially expanded configuration. In the depicted example, the lower cell portions 230L have a quadrilateral shape when the frame 200 is in the radially expanded configuration. When an outer skirt (similar to 18, 106, 156) is attached to the support members 250, the lower cell portions 230L can be covered by the outer skirt, whereas the upper cell portions 230U can provide an opening for coronary access.

In the example depicted in FIGS. 4A-4D, the joint portion 254 of each support member 250 is configured to move axially toward the outflow end 218 (thus farther away from the inflow end 216) when the frame 200 is radially compressed (thus allowing the corresponding arm portions 252 to fold) and moved axially toward the inflow end 216 (thus farther away from the outflow end 218) when the frame 200 is radially expanded (thus allowing the corresponding arm portions 252 to unfold).

FIGS. 5A-5C depict another radially expandable and compressible frame 260 which can be incorporated in any of the prosthetic valves (for example, 10, 100, 150) described herein. Similar to 200, the frame 260 include four rows (for example, 222, 224, 226, 228) of angled struts 202 and a plurality of axial frame members 210, which includes three commissure supports 232 having respective commissure windows 236 and six axial posts 234 circumferentially distributed between the commissure supports 232. Likewise, the rows of angled struts 202 and the axial frame members 210 can define a row of first cells 230, a row of second cells 238, and a row of third cells 240.

The frame 260 also includes a plurality of support members 250 extending between the axial frame members 210. Similarly, each support member 250 comprises two arm portions 252 connected by a joint portion 254, and each arm portion 252 is connected to a respective axial frame member 210 at a corresponding connecting point 258. In the example depicted in FIGS. 5A-5C, each arm portion 252 is configured to be substantially straight. Likewise, the support members 250 can be configured to be narrower than the axial frame members 210 and the angled struts 202 in any one of the rows (for example, 222, 224, 226, 228).

Unlike the example of FIGS. 4A-4D where the connecting points 258 overlap with the second ends 214 of the axial frame members 210, in the example depicted in FIGS. 5A-5C, the connecting points 258 are axially located between the first ends 212 and second ends 214 of the axial frame members. Thus, only the second row 224 of angled struts connect the second ends 214 of the axial frame members 210.

As shown in FIGS. 5A-5C, for support members 250 connected to commissure supports 232, the corresponding connecting points 258 can be located between the first ends 212 and the commissure windows 236, that is, the connecting points 258 can be axially spaced apart from the commissure windows 236. In other examples, the connecting points 258 can be located axially along the commissure windows 236. The commissure supports 232 have less material or mass along the portions of the supports 232 forming the window. Thus, in certain cases, placing the connecting points 258 away from the commissure windows 236 may improve the stability of the connecting points 258 (for example, reduce the likelihood that the connecting points bending inwardly and squeezing commissure tabs when expanding the frame 260).

In some examples, as depicted in FIGS. 5A-5C, the axial posts 234 can include apertures 235 (similar to 132 of FIG. 3) configured to allow a suture to extend through so as to fasten the outer skirt to the axial posts 234. In the depicted example, the apertures 235 are located at about the second ends 214 of the axial posts 234. In other examples, the apertures 235 can be located at positions between the first ends 212 and second ends 214 of the axial posts 234. In still another example, the apertures 235 can be located at about the first ends 212 of the axial posts 234. In the depicted example, each axial post 234 has one aperture 235. In other examples, some axial posts 234 can have more than one aperture 235 or have no aperture 235. In still other examples, some of the commissure supports 232 can also have apertures 235, which can be located above and/or below the commissure windows 236.

Similarly, the support members 250 can divide the first cells 230 into respective upper cell portions 230U and lower cell portions 230L. In the example depicted in FIG. 5B, the upper cell portions 230U are smaller than the lower cell portions 230L when the frame 260 is in the radially expanded configuration. In other examples, the upper cell portions 230U can be larger than or have about the same size as the lower cell portions 230L when the frame 260 is in the radially expanded configuration. In the depicted example, the upper cell portions 230U have a hexagonal shape and the lower cell portions 230L have an irregular hexagonal shape when the frame 260 is in the radially expanded configuration.

In the example depicted in FIGS. 5A-5C, the joint portion 254 of each support member 250 extends downward (that is, toward the inflow end), and is configured to move axially toward the inflow end 216 (thus away from the outflow end 218) when the frame 260 is radially compressed (thus allowing the corresponding arm portions 252 to fold) and moved axially toward the outflow end 218 (thus away from the inflow end 216) when the frame 260 is radially expanded (thus allowing the corresponding arm portions 252 to unfold). As shown in FIG. 5C, when folded, the two arm portions 252 of each support member 250 can be configured to extend substantially parallel to and between two immediately adjacent axial frame members 210.

The connecting points 258 can be configured to be at other locations of the axial frame members 210.

As one example, FIG. 7A shows a column of a frame 270, where a support member 250 connects a commissure support 232 and an axial post 234. The connecting point 258 on the commissure support 232 is located between the second end 214 and the commissure window 236. The connecting point 258 on the axial post 234 is located above, and adjacent to, the second end 214, which has an aperture 235. The joint portion 254 of the support member 250 extends upward (pointing toward the outflow end 218). Thus, when the frame 270 is radially compressed, the joint portion 254 can move toward the outflow end 218 (and away from the inflow end 216) while the two arm portions 252 fold between the commissure support 232 and the axial post 234.

As another example, FIG. 7B shows a column of another frame 280, where a support member 250 connects a commissure support 232 and an axial post 234. In this example, the connecting points 258 on both commissure support 232 and axial post 234 are at about mid-points of the respective first ends 212 and second ends 214. In FIG. 7B, the commissure window 236 can be configured to be smaller than the one depicted in FIG. 7A. In the depicted example, the commissure window 236 is located above the corresponding connecting point 258. In other examples, the commissure window 236 can be located below the corresponding connecting point 258. In the depicted example, the joint portion 254 of the support member 250 extends upward and can move toward the outflow end 218 when the frame 280 is radially compressed. In other examples, the joint portion 254 can extend downward and move toward the inflow end 216 when the frame 280 is radially compressed.

As yet another example, FIG. 7C shows a column of another frame 290, where a support member 250 connects a commissure support 232 and an axial post 234. In this example, the connecting points 258 on both commissure support 232 and axial post 234 are at about the respective first ends 212. The joint portion 254 of the support member 250 extends toward and can move toward the inflow end 216 when the frame 290 is radially compressed.

In certain examples, as depicted in FIGS. 7A-7C, the attachment portions or end portions 256 of each arm portion 252 can have a curved shape, and each arm portion 252 can be connected to a corresponding commissure support 232 or axial post 234 via the curved attachment portion 256. The curvature of the attachment portions 256 can be configured to facilitate folding of the support members 250. For example, when the joint portion 254 of the support member 250 extends upward (see, for example, FIGS. 7A-7B), the corresponding attachment portions 256 can form an arc of a circle whose center is located below or more upstream of the connecting points 258. Conversely, when the joint portion 254 extends downward (see, for example, FIG. 7C), the corresponding attachment portions 256 can form an arc of a circle whose center is located above or more downstream of the connecting points 258.

It is to be understood that the examples depicted in FIGS. 7A-7C are not exhaustive. In various examples, the connecting points 258 can be located at any part of the axial frame members 210. In various examples, the connecting points 258 can be axially spaced apart from or aligned with openings (for example, commissure windows 236 and/or apertures 235) on the axial frame members 210. In various examples, the joint portions 254 of the support members 250 can extend toward the inflow end 216 or outflow end 218. Additionally, when the frame is in the radially expanded configuration, the angle formed at the joint portions 254 and between the pairs of corresponding arm portions 252 can also vary, for example, between 90 and 180 degrees, between 100 and 170 degrees, between 110 and 160 degrees, between 120 and 150 degrees, between 130 and 140 degrees, etc.

Such design variations allow the connecting points 258 and/or the joint portions 254 to be positioned at different heights relative to the axial frame members 210. Thus, it allows the outer skirt, which can be attached to the support members 250, to cover different area of the first cells 230.

In certain examples, the joint portions 254 can be closer to the outflow end 218 than the second ends 214 of the axial frame members 210 when the frame is in the radially expanded configuration. In certain examples, the joint portions 254 can be axially aligned with a midpoint between the first ends 212 and the second ends 214 when the frame is in the radially expanded configuration.

In certain examples, the connecting points 258 can be axially closer to the first ends 212 than a midpoint between the first ends 212 and the second ends 214. In certain examples, a midpoint between the first ends 212 and the second ends 214 can be axially closer to the first ends 212 than the connecting points 258.

In certain examples, the joint portions 254 of the support members 250 can be axially closer to the second ends 214 than the connecting points 258 when the frame is in the radially expanded configuration. In certain examples, the joint portions 254 of the support members 250 can be axially closer to the first ends 212 than the connecting points 258 when the frame is in the radially expanded configuration. In one specific example, the joint portions 254 can be axially aligned with the first ends 212 of the axial frame members 210 when the frame is in the radially expanded configuration.

In some examples, an axial distance H (see, for example, FIGS. 4B and 5B) between the inflow end 216 of a frame and the joint portions 254 of the support members 250 can be at least 10 mm when the frame is in the radially expanded configuration. In some examples, the axial distance H can be between one third and two thirds of the frame height (that is, the axial distance measured between the inflow end 216 and the outflow end 218) when the frame is in the radially expanded configuration.

When an outer skirt (similar to 18, 106, 156) is disposed on the outer surface of the frame and an outflow edge portion (for example, 122, 152) of the outer skirt is connected to the support members 250, the outer skirt can extend from the inflow end 216 of the frame to the support members, thus covering at least a lower portion of the first cells 230 (for example, the lower cell portions 230L). Because the support members 250 extends across a mid-portion of the first cells 230, the support members 250 can provide additional structural support of the outer skirt. In some instances, such additional structure support of outer skirt can enhance the connection of the outflow edge of the skirt to the frame and may help reduce the likelihood that portions of the outflow edge portion of the outer skirt protrude inwardly through the first cells 230 and contact the leaflets 104.

In some examples, the outflow edge portion of the outer skirt can be axially located between the first ends 212 and the second ends 214 of the axial frame members 210. In some examples, the outflow edge portion of the outer skirt can be axially closer to the outflow end 218 of the frame than the second ends 214 of the axial frame members 210. In one specific example, the outflow edge portion of the outer skirt can be axially aligned with a mid-point between the first ends 212 and the second ends 214 of the axial frame members 210. In another specific example, the outflow edge portion of the outer skirt can be axially aligned with the first ends 212 of the axial frame members 210.

To assemble a prosthetic valve, an annular frame having rungs of support members, as described above, can be prepared. In certain examples, the frame can be constructed by forming individual components (for example, the angled struts, the axial frame members, the support members, etc.) and then mechanically assembling and connecting the individual components together. In other examples, the frame can be constructed from a single piece of material (for example, Nitinol, stainless steel, cobalt-chromium alloy, etc.), such as in the form of a tube. The plurality of cells, struts, and support members can be formed by removing portions (for example, via laser cutting, electroforming, physical vapor deposition, etc.) of the single piece of material.

In certain examples, the frame can be initially constructed to include rows of angled struts and axial frame members, and then the support members can be added (for example, welded) to connect the axial frame members.

Once the frame is formed, a valvular structure comprising a plurality of leaflets (similar to 40 or 104) can be mounted within the frame. In certain examples, an inner skirt (similar to 16) can be attached to an inner surface of the frame, and the inner skirt can be configured to be between leaflets and the inner surface of the frame. An outer skirt (similar to 18, 106, 156) can be mounted on an outer surface of the frame. An inflow edge portion of the outer skirt can be secured to inflow struts of the frame, and an outflow edge portion of the outer skirt can be connected to the support members, for example, via stitches. In certain cases, the outflow edge portion of the outer skirt can also be connected to at least some of the axial frame members, for example, via stitches.

### Exemplary Frames Having Strengthened Outflow End Portions

In some circumstances, after receiving the transcatheter aortic valve replacement surgery, a patient may need to undergo a post-implantation procedure that requires open-heart surgery involving a heart-lung machine. In open-heart surgery, an aortic cross-clamp is often used to clamp the aorta to separate the systemic circulation from the outflow of the heart. If the aortic cross-clamp is placed at a position corresponding to the implanted prosthetic valve, there is a possibility that the aortic cross-clamp may deform the prosthetic valve, especially if the valve has a plastically deformable frame.

As described herein, any of the prosthetic valves (for example, 10, 100, 150) described herein can be strengthened to have improved crush resistance against the compressive forces applied by an aortic cross-clamp. This can be accomplished, for example, by strengthening an outflow end portion of the frame that is downstream of the leaflets. Limiting the strengthening to the outflow end portion of the frame is desirable because further strengthening other portions of the frame may require inflating the balloon in a manner that would exert greater forces to expand the frame, which may damage or even tear the leaflets. Such risk can be reduced if the strengthened frame portion is located downstream of the leaflets.

In some examples, a prosthetic valve can be strengthened by strengthening the outflow struts of the frame, for example, by thickening and/or widening the outflow struts. As an example, the first row of struts 111 of the frame 102 can have a larger width than other rows (121, 123, 115) of struts. As another example, the first row of struts 222 of the frame 200 can have a larger width than other rows (224, 226, 228) of struts. In certain circumstances, increasing the width of the outflow struts by 15% can increase the strength of the outflow struts by about 50%, and increasing the width of the outflow struts by 25% can increase the strength of the outflow struts by about 100%. Additionally, and/or alternatively, the outflow struts can be strengthened by using a metal and/or alloy material that is stronger than other parts of the frame.

In some examples, the outflow end portion of the frame can be strengthened by adding rungs of support members, similar to the examples shown in FIGS. 5A-5B and FIG. 7C. For example, rungs of support members 250 can be added between the axial frame members 210, with the connecting points 258 adjacent the outflow struts 208 (similar to the examples of FIGS. 5A-5B) or overlapping with the first ends 212 of the axial frame members 210 (similar to the examples of FIG. 7C). Thus, in lieu of or in addition to serving as supporting arms for attachment of the outer skirt, the rungs of support members 250 depicted in FIGS. 5A-5B and FIG. 7C can also enhance the strength of the outflow end portion of the frame. In some examples, the outflow end portion of the frame can be strengthened by both incorporating the rungs of support members similar to FIGS. 5A-5B and FIG. 7C, and/or by thickening and/or widening the outflow struts 208 as described above.

In some examples, the outflow end portion of the frame can be strengthened by adding one or more optional axial support struts bridging the rungs of support members and the outflow struts. For example, FIG. 4D depicts an axial support strut 225 (shown in dashed lines to denote it is optional) connecting between the joint portion 254 of a support member 250 and the apex portion 205 of an outflow strut 208. In such case, the axial support strut 225 can further divide the upper cell portion 230U of the first cell 230 into two side-by-side half portions. In some examples, the axial support strut 225 can bridge each pair of a support member 250 and an outflow strut 208 (that is, each cell portion 230U of the frame includes an axial support strut 225). In other examples, the axial support struts 225 can bridge selected pairs of support members 250 and outflow struts 208 (for example, every other cell portion 230U can include an axial support strut 225).

In the examples depicted in FIGS. 5A-5B and FIG. 7C, the connecting points 258 for the rungs of support members 250 are located on the axial frame members 210 such that the rungs of support members 250 are upstream of the outflow struts 208 and divide the outflow cells 230 into upper cell portions 230U and lower cell portions 230L. In other examples, the outflow end portion of the frame can be strengthened by adding rungs of support members downstream of the outflow struts, as described below.

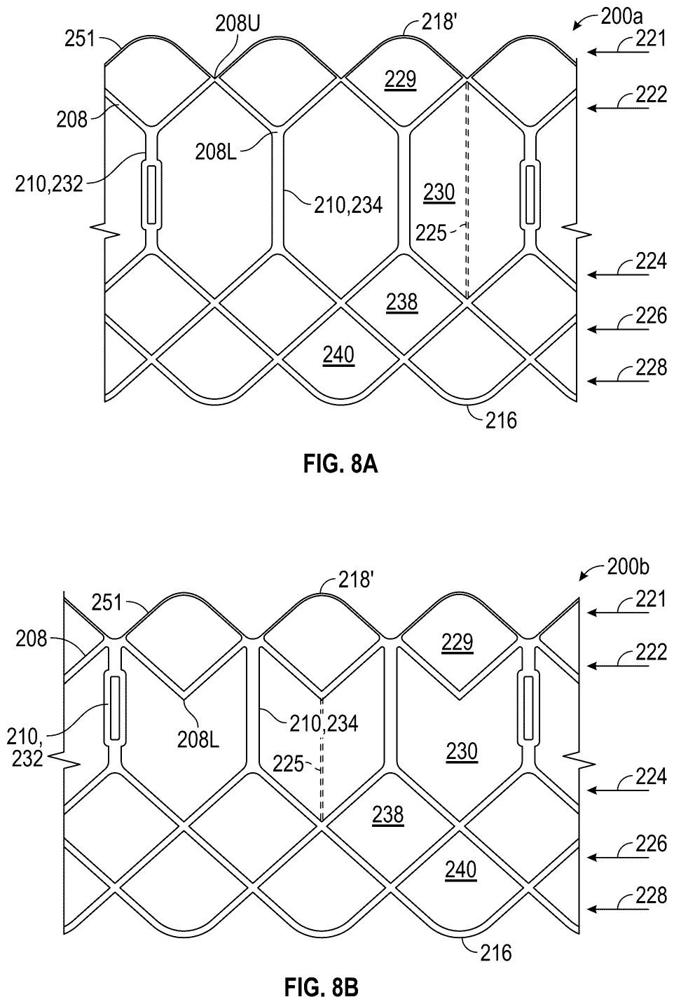

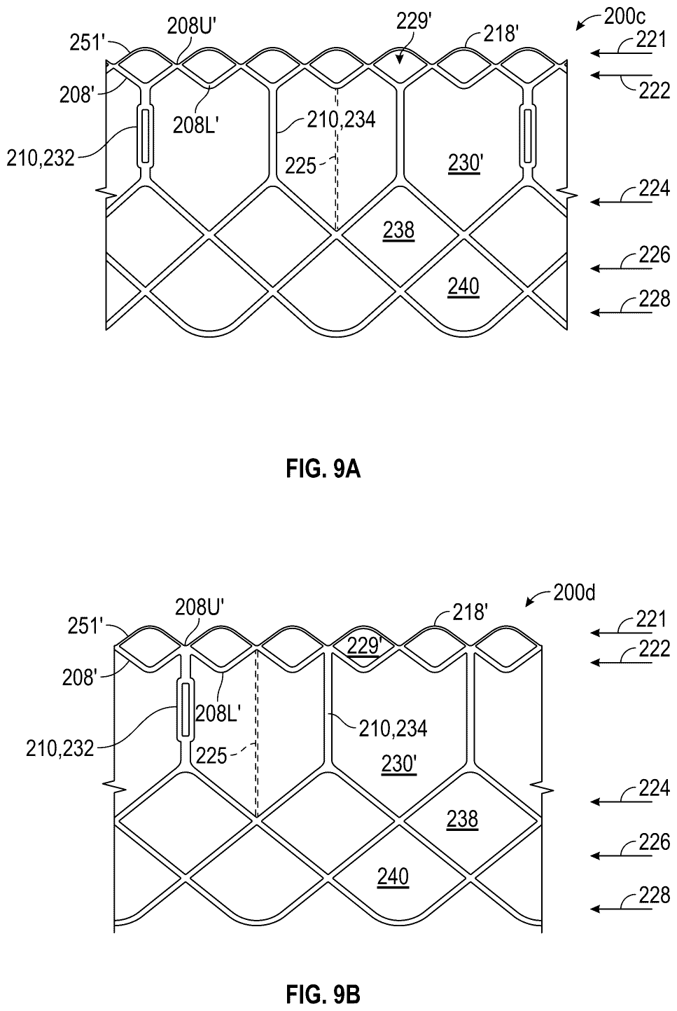

FIGS. 8A-8B and 9A-9B depict additional examples of frames 200a, 200b, 200c, and 200d, each of which can be incorporated in any of the prosthetic valves (for example, 10, 100, 150) described herein. Similarly, each frame 200a, 200b, 200c, or 200d can be moved between a radially compressed configuration and a radially expanded configuration.

The frame 200a depicted in FIG. 8A is similar to the frame 200, 260, 270, 280, and 290 described above, except for the location of the rungs of support members. As shown, the frame 200a has rungs of support members 251 that are connected to and located downstream of the outflow struts 208. As a result, an outflow terminal end 218′ of the frame 200a is defined by the rungs of support members 251, instead of the outflow struts 208. Specifically, the outflow struts 208 are joined together at alternating upper ends 208U and lower ends 208L. The upper ends 208U are axially located between the lower ends 208L and the outflow end 218′. In other words, the lower ends 208L are upstream of the upper ends 208U, which are upstream of the outflow ends 218′. Each of the axial frame members 210 (including commissure supports 232 and axial posts 234) is connected to one of the lower ends 208L in this example.

The frame 200b depicted in FIG. 8B is identical to 200a except that the axial frame members 210 (including commissure supports 232 and axial posts 234) are connected to the upper ends 208L.

When the rungs of support members 251 are located downstream of the outflow struts 208, the rungs of support members 251 can also be deemed as a row of angled struts. Thus, the frame 200a or 200b has at least five rows of angled struts: a first row 221 of angled struts defined by the rungs of support members 251, a second row 222 of angled struts defined by the outflow struts 222, a third row 224 of angled struts upstream of the second row 222, a fourth row 226 of angled struts upstream of the third row 224, and a fifth row 228 of angled struts upstream of the fourth row 226. The rows 222, 224, 226, and 228 respectively correspond to the same numbered rows of FIG. 4B. In the depicted example, the axial frame members 210 (including commissure supports 232 and axial posts 234) connect the second row 222 and the third row 224 of angled struts, and the fifth row 228 of angled struts define the inflow end 216. In other examples, the frame 200a or 200b can have more than five rows of angled struts (for example, additional rows of angled struts can be added upstream of the fifth row 228).

Similarly, the added rungs of support members 251 can enhance the strength of the outflow end portion of the frame 200a or 200b. In some examples, the outflow end portion of the frame 200a or 200b can be further strengthened by thickening and/or widening the outflow struts 208 as described above. For example, the angled struts of the second row 222 (the outflow struts 208) can be wider than the angled struts of other rows (221, 224, 226, and 228). In some examples, the angled struts of the first row 221 (the rungs of support members 251) can have a smaller width than the angled struts of other rows (222, 224, 226, and 228). In some examples, the angled struts of the first row 221 can have the same width as the angled struts of the second row 222, the third row 224, the fourth row 226, and/or the fifth row 228.

As shown in FIGS. 8A-8B, the frame 200a or 200b can have at least four rows of open cells: the first row 221 of angled struts and the second row 222 of angled struts can define a plurality of first cells 229; the second row 222 of angled struts, the third row 224 of angled struts, and the plurality of axial frame members 210 can define a plurality of second cells 230; the third row 224 of angled struts and the fourth row 226 of angled struts can define a plurality of third cells 238; the fourth row 226 of angled struts and the fifth row 228 of angled struts can define a plurality of fourth cells 240. The cells 230, 238, and 240 respectively correspond to the same numbered cells of FIG. 4B.

In the depicted examples, the first cells 229 have a substantially diamond shape when the frame 200a or 200b is in the radially expanded configuration. The shapes of the cells 230, 238 and 240 respectively match the same numbered cells of FIG. 4B. When the frame 200a or 200b is in the radially expanded configuration, the second cells 230 have a larger area (and a larger axial length) than other cells (229, 238, and 240). In this example, the first cells 229 has the same number as the second cells 230, the third cells 238, and the fourth cells 240 (for examples, 9 cells each for 229, 230, 238, and 240).

In some examples, the outflow end portion of the frame 200a or 200b can be further strengthened by adding one or more axial support struts bridging the second row 222 and third row 224 of angled struts. For example, an axial support strut 225 (shown in dashed lines to denote it is optional) can connect and extend between a joint of two angled struts in the second row 222 (for example, 208U in FIG. 8A or 208L in FIG. 8B) and an axially aligned joint of two angled struts in the third row 224 such that the corresponding second cell 230 can be divided into two side-by-side half portions. In some examples, each second cell 230 can include an axial support strut 225. In other examples, selected second cells 230 (for example, every other second cell 230) can include the axial support struts 225.

The frame 200c depicted in FIG. 9A is similar to the frame 200a described above, except for the arrangement of outflow struts and rungs of support members. In contrast to the frame 200a which has two angled outflow struts 208 bridging each pair of adjacent axial frame members 210, the frame 200c has four angled outflow struts 208′ extending between each pair of adjacent axial frame members 210. The outflow terminal end 218′ of the frame 200c is defined by rungs of support members 251′, which are connected to and located downstream of the outflow struts 208′. As shown, the outflow struts 208′ are joined together at alternating upper ends 208U′ and lower ends 208L′, and the upper ends 208U′ are axially located between the lower ends 208L′ and the outflow end 218′. In the depicted example, the axial frame members 210 (including commissure supports 232 and axial posts 234) are connected to every other one of the lower ends 208L′.

The frame 200d depicted in FIG. 9B is identical to 200c except that each one of the axial frame members 210 (including commissure supports 232 and axial posts 234) is connected to every other one of the upper ends 208U.

Similarly, the frame 200c or 200d has at least five rows of angled struts: a first row 221 formed by the rungs of support members 251′, a second row 222 formed by the outflow struts 208′, a third row 224 upstream of the second row 222, a fourth row 226 upstream of the third row 224, and a fifth row 228 upstream of the fourth row 226.

In this example, the added rungs of support members 251′ can enhance the strength of the outflow end portion of the frame 200c or 200d. In some examples, the outflow end portion of the frame 200c or 200c can be further strengthened by thickening and/or widening the outflow struts 208′ as described above. For example, the struts of the second row 222 of angled struts (the outflow struts 208′) can be wider than other rows (221, 224, 226, and 228) of angled struts. In some examples, the struts of the first row 221 of angled struts (the rungs of support members 251′) can have a smaller width than other rows (222, 224, 226, and 228) of angled struts. In some examples, the struts of the first 221 of angled struts can have the same width as the struts of the second row 222, the third row 224, the fourth row 226, and/or the fifth row 228.

Similarly, the frame 200c or 200d has at least four row of cells: first cells 229′ formed by the first row 221 and second row 222 of angled struts, second cells 230′ formed by the second row 222 and third row 224 of angled struts and the axial frame members 210, third cells 238 formed by the third row 224 and fourth row 226 of angled struts, and fourth cells 240 formed by the fourth row 226 and fifth row 228 of angled struts.

In FIGS. 9A-9B, the third and fourth cells 238, 240 respectively correspond to the same numbered cells of FIGS. 8A-8B. But the first and second cells 229′, 230′ can differ in size, shape, and/or number from the corresponding numbered cells of FIGS. 8A-8B. For example, while there are the same number of second cells 230′ as the third cells 238 and fourth cells 240, the number of first cells 229′ is twice the number of second cells 230′. In one specific example, the number of first cells 229′ is 18 and the number of second cells 230′ is 9. Additionally, when the frame 200c or 200d is in the radially expanded configuration, the first cells 229′ have a smaller area (for example, less than 50%) than the third cells 238 and/or the fourth cells 240.

It is to be understood that the frame configurations shown in FIGS. 8A-8B and 9A-9B are merely exemplary, and the frame can have other configurations based on the principle described herein. For example, the number (N) of angled outflow struts connecting each pair of adjacent axial frame members 210 can be other than 2 (as in FIGS. 8A-8B) or 4 (as in FIGS. 9A-9B), such as 3, 5, 6, 7, 8, etc. Rungs of support members, which define the outflow terminal end of the frame, can be connected to, and located downstream of these outflow struts, thereby forming a plurality of first cells. The ratio (R) between the number of first cells and the number of second cells can be N:2. For example, the ratio R is 3:2 if N=3,or 3:1 if N=6, etc.

In some examples, the outflow end portion of the frame 200c or 200d can be further strengthened by adding one or more axial support struts bridging the second row 222 and third row 224 of angled struts. For example, an axial support strut 225 (shown in dashed lines to denote it is optional) can connect and extend between a joint of two angled struts in the second row 222 (for example, 208L′ in FIG. 9A or 208U′ in FIG. 9B) and an axially aligned joint of two angled struts in the third row 224 such that the corresponding second cell 230′ can be divided into two side-by-side half portions. In some examples, each second cell 230′ can include an axial support strut 225. In other examples, selected second cells 230′ (for example, every other second cell 230′) can include the axial support struts 225.

### Exemplary Frames Having Other Configurations of Support Structures

FIGS. 10-19 depict additional examples of frames that can be used in any of the prosthetic valves described herein (for example, 10, 100, 150). As described more fully below, each of the frames shown in FIGS. 10-19 has a support structure which includes two or more angled support arms and at least one axial support member. The support structures in these examples can divide outflow cells of the frame into a plurality of sub-cells. Some of the sub-cells have large areas to facilitate coronary access therethrough. The support structures can prevent or limit leaflets of the prosthetic valve from bulging outwardly through large openings of the outflow cells during valve crimping, and can also prevent or limit native leaflets from protruding inwardly through the outflow cells during cardiac cycles. The support structures can also reduce risk of leaflets abrasion, for example, by eliminating apices formed between angled support arms (for example, joint portion 254). Additionally, the axial support members in the support structures can improve stability of the prosthetic valve and prevent any part of the support structure from protruding outwardly when crimped, thus allowing undisturbed axial movement of the prosthetic valve within a delivery catheter.

FIGS. 10 and 10A-10B show an annular frame 300 for a prosthetic valve, according to one example. Similar to the frame 260 of FIGS. 5A-5C, the frame 300 include four rows (for example, rows 322, 324, 326, 328) of angled struts 302. The first row 322 of angled struts (also referred to as “outflow struts”) define an outflow end 318 and the fourth row 328 of angled struts (also referred to as “inflow struts”) define an inflow end 316 of the frame 300. The frame 300 also includes a plurality of axial frame members 310 bridging the first row 322 and second row 324 of angled struts. In the example depicted in FIG. 10, the axial frame members 310 includes three commissure supports 332 having respective commissure windows 336 and six axial posts 334. Two axial posts 334 are circumferentially distributed between each pair of adjacent commissure supports 332. The first and second rows 322, 324 of angled struts and the axial frame members 310 can define a row of six-sided first cells 330 (also referred to as “outflow cells”). The second and third rows 324, 326 of angled struts can define a row of four-sided second cells 338. The third and fourth rows 326, 328 of angled struts can define a row of four-sided third cells 340 (also referred to as “inflow cells”). In the depicted example, the frame 300 has nine first cells 330, nine second cells 338, and nine third cells 340.

The frame 300 also includes a plurality of support structures 350 connecting the plurality of axial frame members 310 and the second row 324 of angled struts. In the example depicted in FIGS. 10 and 10A-10B, each support structure 350 comprises two angled support arms 352 connecting two immediately adjacent axial frame members 310 and one axial support member 354 bridging the two angled support arms 352 and the second row 324 of angle struts. When the frame 300 is used in a prosthetic valve, an outer skirt (for example, 18, 106, 156) can be attached to the angled support arms 352 of each support structure 350. For example, an outflow edge portion (for example, 122, 152) of the outer skirt can be connected to the angled support arms 352 via sutures or other means.

The angled support arms 352 can be similar to the arm portions 252 of the support member 250 described above. For example, each angled support arm 352 can be connected to a corresponding axial frame member 310 at a connecting point 358. As shown in FIG. 10A, two angled support arms 352 of two adjacent support structures 350 located on opposite sides of an axial frame member 310 can be connected to the axial frame member 310 at the connecting point 358.

Each axial frame member 310 has an upper end 310U connected to the first row 322 of angled struts and a lower end 310L connected to the second row 324 of angled struts. In some examples, as depicted in FIGS. 10 and 10A-10B, the connecting point 358 is axially spaced away from the upper end 310U. In some examples, the connecting point 358 can be closer to the upper end 310U than the lower end 310L. In other examples, the upper end 310U can overlap with the connecting point 358.

For each support structure 350, the two angled support arms 352 and the axial support member 354 are connected at a joint portion 356 so that the two angled support arms 352 and the axial support member 354 can define a Y-shaped configuration. The joint portion 356 can define an upper end of the axial support member 354. As shown in FIG. 10A, each Y-shaped support structure 350 can divide a corresponding outflow cell 330 into three openings or sub-cells: one upper sub-cell 330U and two lower sub-cells 330L. In the depicted example, the frame 300 has nine upper sub-cells 330U and eighteen lower sub-cells 330L. Each upper sub-cell 330U has a width that is twice that of a lower sub-cell 330L.

In the example depicted in FIGS. 10 and 10A-10B, each upper sub-cell 330U is six-sided, formed by two angled strut portions 304 in the first row 322 of angled struts, two angled support arms 352, and two upper portions 312 of two axial frame members 310. In other examples, each upper-cell 330U can be four-sided. For example, when the upper end 310U overlaps with the connecting point 358, each upper sub-cell 330U is bounded by two angled strut portions 304 in the first row 322 of angled struts and two angled support arms 352.

As shown in FIG. 10A, the second row 324 of angled struts are joined together at alternating upper ends 324U and lower ends 324L (the upper ends 324U being closer to the outflow end 318 than the lower ends 324L). The axial support members 354 can be connected to the corresponding lower ends 324L of the second row 324 of angled struts. Thus, the lower ends 324L of the angled struts in the second row 324 can also define the lower ends of the axial support members 354. In other words, the axial support members 354 terminate at the corresponding lower ends 324L.

In some examples, the angled support arms 352 can be substantially parallel to corresponding angled struts in the second row 324 of angled struts. Thus, each of the lower sub-cells 330L can have a parallelogram shape.

Each support structure 350 can be configured to be axially foldable. For example, similar to the arm portions 252 of FIGS. 5A-5C, each pair of angled support arms 352 can fold at the joint portion 356 when the frame 300 moves from the radially expanded configuration (see, for example, FIG. 10A) to the radially compressed configuration (see, for example, FIG. 10B). When folded, the two angled support arms 352 of each support structure 350 can extend substantially parallel to and between two immediately adjacent axial frame members 310. In addition, the axial support members 354 can move axially when the frame 300 moves from the radially expanded configuration to the radially compressed configuration.

Connecting between the angled support arms 352 and the second row 324 of angled struts, the axial support members 354 can eliminate free apices where the support arms 352 intersect with each other, thereby reducing the risk of leaflet abrasion. The Y-shaped support structure 350 can also be confined within a plane defined by the corresponding outflow cell 330 (for example, any part of the support structure 350 can be prevented from protruding outwardly when the frame 300 is crimped), thereby improving stability of the frame 300 (and the corresponding prosthetic valve). Additionally, the Y-shaped support structure 350 can function as a barrier to prevent leaflets of the prosthetic valve from bulging outwardly and/or prevent native leaflets from protruding inwardly through the outflow cells 330.

In some examples, the angled support arms 352 are narrower than the angled struts 302 in any row (for example, 322, 324, 326, 328) of angled struts. In some examples, the axial support members 354 are narrower than the axial frame members 310 (including the commissure supports 332 and the axial posts 334).

In some examples, the angled support arms 352 are narrower than the axial support members 354. In other examples, the angled support arms 352 can have about the same width as the axial support members 354.

FIG. 10A also illustrates relative positions of a leaflet (for example, 40 or 104) and the frame 300 after mounting the leaflet to the frame 300 (the outer perimeter of the leaflet is shown in dashed lines), according to one example. As shown, an outflow edge 42 of the leaflet can extend across three outflow cells 330. The axial position of the outflow edge 42 can be anywhere between upper ends of the commissure windows 336 and upper ends 310U of the axial frame member 310. Each commissure tab 44 of the leaflet can be paired with an adjacent commissure tab of an adjacent leaflet to form a commissure of a leaflet assembly that is inserted into a corresponding commissure window 336. A cusp edge 46 of the leaflet (which is below the commissure tabs 44 and opposite to the outflow edge 42) can have a generally V-shaped contour. The cusp edge 46 can extend along and be attached to four angled struts (for example, 324a, 324b, 326a, and 326b) respectively located on the second row 324 and third row 326, and two angled strut portions (for example, 306a and 306b) located on the fourth row 328. An apex region 48 of leaflet can align with a curved apex portion 307 (similar to 207 of FIGS. 5A-5B) of an inflow strut at the inflow end 316 of the frame 300.

FIG. 11 shows a section of an annular frame 400 for a prosthetic valve wherein the frame section is shown in a flattened configuration, according to another example. The full frame 400 includes three such frame sections. The frame 400 is similar to the frame 300 except that the frame 400 has five rows (for example, rows 422, 424, 426, 428, 429) of angled struts 402. In some examples, the frame 400 can have a larger diameter than the frame 300 when they are both in the radially expanded configuration. In some examples, a frame similar to 300 and 400 can have more than five rows of angled struts.

In the example depicted in FIG. 11, the first row 422 of angled struts define an outflow end 418 and the fifth row 429 of angled struts define an inflow end 416 of the frame 400. Similar to the frame 300, the frame 400 includes a plurality of axial frame members 410 bridging the first row 422 and second row 424 of angled struts. For example, the axial frame members 410 can include three commissure supports 432 having respective commissure windows 436 and nine axial posts 434. Three axial posts 434 can be circumferentially distributed between each two adjacent commissure supports 432. The first and second rows 422, 424 of angled struts and the axial frame members 410 can define a row of six-sided outflow cells 430. The fourth and fifth rows 428, 429 of angled struts can define a row of four-sided inflow cells 442. The second, third, and fourth rows 424, 426, 428 of angled struts can define two intermediate rows of four-sided cells 438, 440. In this example, the number of cells in each row (430, 438, 440, 442) is twelve.

The frame 400 also includes a plurality of support structures 450 that are similar to the support structures 350 depicted in FIGS. 10 and 10A-10B. For example, each support structure 450 has two angled support arms 452 connecting two immediately adjacent axial frame members 410 and one axial support member 454 bridging the two angled support arms 452 and the second row 424 of angle struts. Similarly, each support structure 450 can divide an outflow cell 430 into one upper sub-cell 430U and two lower sub-cells 430L. As a result, the frame 400 has 12 upper sub-cells 430U and 24 lower sub-cells 430L.

FIG. 11 also illustrates relative positions of a leaflet (for example, 40 or 104) and the frame 400 after mounting the leaflet to the frame 400 (the outer perimeter of the leaflet is shown in dashed lines), according to one example. As shown, the outflow edge 42 of the leaflet can extend across four outflow cells 430. The axial position of the outflow edge 42 can be anywhere between upper ends of the commissure windows 436 and upper ends 410U of the axial frame member 410. Each commissure tab 44 of the leaflet can be paired with an adjacent commissure tab of an adjacent leaflet to form a commissure of a leaflet assembly that is inserted into a corresponding commissure window 436. The cusp edge 46 of the leaflet can extend along and be attached to six angled struts (for example, 424a, 424b, 426a, 426b, 428a, and 428b) respectively located on the second row 424, third row 426 and fourth row 428, and two angled strut portions (for example, 406a and 406b) located on the fifth row 429. The apex region 48 of leaflet can align with a curved apex portion 407 (similar to 207 of FIGS. 5A-5B) of an inflow strut at the inflow end 416 of the frame 400.

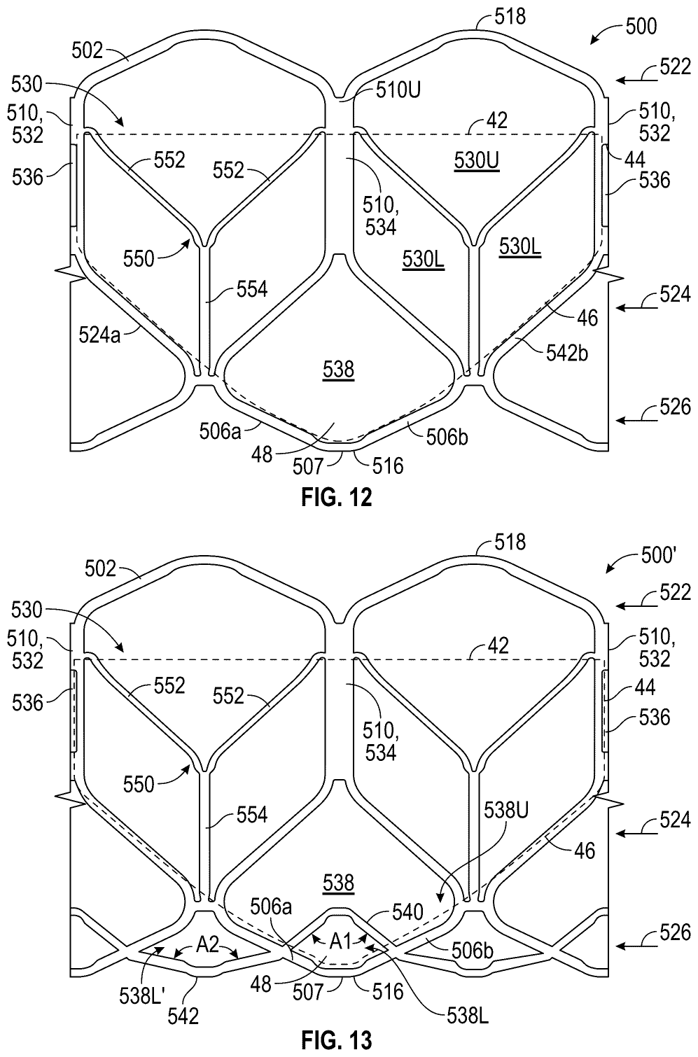

FIG. 12 shows a section of an annular frame 500 for a prosthetic valve shown in a flattened configuration, according to another example. The full frame 500 includes three such sections. The frame 500 is similar to the frame 300 except that the frame 500 has only three rows (for example, 522, 524, 526) of angled struts 502. In some examples, the frame 500 can have a smaller diameter than the frame 300 when they are both in the radially expanded configuration.

In the example depicted in FIG. 12, the first row 522 of angled struts define an outflow end 518 and the third row 526 of angled struts define an inflow end 516 of the frame 500. Similar to the frame 300, the frame 500 includes a plurality of axial frame members 510 bridging the first row 522 and second row 524 of angled struts. For example, the axial frame members 510 can include three commissure supports 532 having respective commissure windows 536 and three axial posts 534. Each axial post is circumferentially positioned between two adjacent commissure supports 532. The first and second rows 522, 524 of angled struts and the axial frame members 510 can define a row of six-sided outflow cells 530. The second and third rows 524, 526 of angled struts can define a row of four-sided inflow cells 538. In this example, the frame 500 has six outflow cells 530 and six inflow cells 538.

The frame 500 also includes a plurality of support structures 550 that are similar to the support structures 350 depicted in FIGS. 10 and 10A-10B. For example, each support structure 550 has two angled support arms 552 connecting two immediately adjacent axial frame members 510 and one axial support member 554 bridging the two angled support arms 552 and the second row 524 of angle struts. Similarly, each support structure 550 can divide an outflow cell 530 into one upper sub-cell 530U and two lower sub-cells 530L. As a result, the frame 500 has six upper sub-cells 530U and twelve lower sub-cells 530L.

FIG. 12 also illustrates relative positions of a leaflet (for example, 40 or 104) and the frame 500 after mounting the leaflet to the frame 500 (the outer perimeter of the leaflet is shown in dashed lines), according to one example. As shown, the outflow edge 42 of the leaflet can extend across two outflow cells 530. The axial position of the outflow edge 42 can be anywhere between upper ends of the commissure windows 536 and upper ends 510U of the axial frame member 510. Each commissure tab 44 of the leaflet can be paired with an adjacent commissure tab of an adjacent leaflet to form a commissure of a leaflet assembly that is inserted into a corresponding commissure window 536. The cusp edge 46 of the leaflet can extend along and be attached to two angled struts (for example, 524a, 524b) in the second row 524 and two angled strut portions (for example, 506a and 506b) in the second row 526. The apex region 48 of leaflet can align with a curved apex portion 507 (similar to 207 of FIGS. 5A-5B) of an inflow strut at the inflow end 516 of the frame 500.

FIG. 13 shows another example of a section of a frame 500′ that is similar to the frame 500, except that the frame 500′ includes additional angled support members 540, 542 interconnected with the third row of angled struts 526. Six of the angled support members 540 are positioned with the inflow cells 538, each bridging two angled strut portions (for example, 506a and 506b) of a corresponding inflow strut. The other six angled support members 542 are positioned outside the inflow cells 538, each bridging an angled strut portion (for example, 506a or 506b) of one inflow strut and an adjacent angled strut portion of an adjacent inflow strut. Each angled support member 540 has an arc angle A1 pointing toward the inflow end 516, and each angled support members 542 has an arc angle A2 pointing toward the outflow end 518. When the frame 500′ is in the radially expanded configuration, the arc angle A1 can be smaller than the arc angle A2. Each angled support member 540 can divide the corresponding inflow cell 538 into an upper sub-cell 538U and a lower sub-cell 538L. Each angled support member 542, by bridging two adjacent angled strut portions, can form an additional lower sub-cell 538L′.

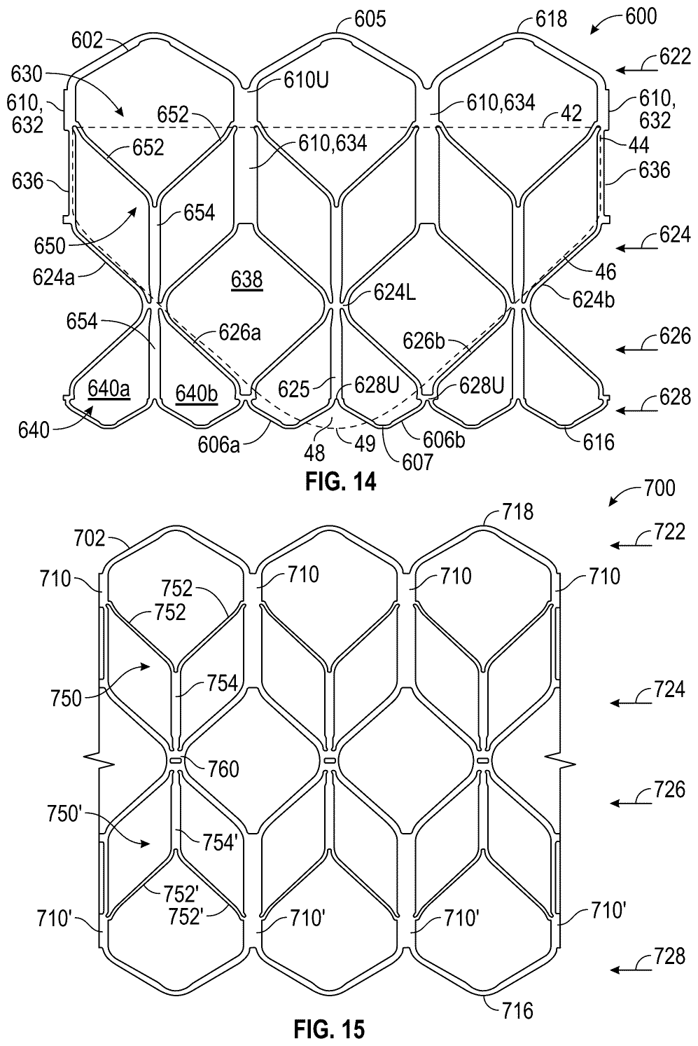

FIG. 14 shows another one-third section of an annular frame 600 for a prosthetic valve, according to another example. Similar to the frame 300, the frame 600 has four rows (for example rows 622, 624, 626, 628) of angled struts 602, wherein the first row 622 of angled struts define an outflow end 618 and the fourth row 628 of angled struts define an inflow end 616 of the frame 600. Similarly, the frame 600 includes a plurality of axial frame members 610 (for example, three commissure supports 632 having respective commissure windows 636 and six axial posts 634) bridging the first row 622 and second row 624 of angled struts. The first and second rows 622, 624 of angled struts and the axial frame members 610 can define a row of six-sided outflow cells 630. The second and third rows 624, 626 of angled struts can define an intermediate row of four-sided cells 638. The third and fourth rows 626, 628 of angled struts can define a row of six-died inflow cells 640.

The frame 600 also includes a plurality of support structures 650. Each support structure 650 has two angled support arms 652 connecting two immediately adjacent axial frame members 610 and one axial support member 654 bridging the two angled support arms 652 and the second row 624 of angle struts. Unlike the frame 300 in which the axial support members 354 terminate at corresponding lower ends 324L of the angled struts in the second row 324, the axial support members 654 of the frame 600 extend further downwardly (toward the inflow end 616) and bridge the second row 624 of angled struts and the fourth row 628 of angled struts (that is, the inflow struts). As a result, each axial support member 654 can divide a corresponding six-sided inflow cell 640 into two four-sided sub-cells 640a, 640b. The inflow struts (at the fourth row 628) of the frame 600 form a zig-zag pattern and define a plurality of upper ends 628U downstream of the inflow end 616. The axial support members 654 of the support structures 650 connect lower ends 624L of the second row 624 of angled struts to the corresponding upper ends 628U of the inflow struts. Each of the remaining upper ends 628U of the inflow struts is connected to two angled struts in the third row 626 of angled struts. In this example, ratio of the number of apices 605 defined by the outflow struts to the number of apices 607 defined by the inflow struts is 1:2.

FIG. 14 also illustrates relative positions of a leaflet (for example, 40 or 104) and the frame 600 after mounting the leaflet to the frame 600 (the outer perimeter of the leaflet is shown in dashed lines), according to one example. As shown, the outflow edge 42 of the leaflet can extend across three outflow cells 630. The axial position of the outflow edge 42 can be anywhere between upper ends of the commissure windows 636 and upper ends 610U of the axial frame member 610. Each commissure tab 44 of the leaflet can be paired with an adjacent commissure tab of an adjacent leaflet to form a commissure of a leaflet assembly that is inserted into a corresponding commissure window 636. The cusp edge 46 of the leaflet can extend along and be attached to at least four angled struts (for example, struts 624a, 624b, 626a, and 626b) respectively located on the second row 624 and third row 626. In some examples, the cusp edge 46 of the leaflet can also be attached to two angled strut portions (for example, struts 606a, 606b) of the inflow struts. Alternatively, the cusp edge 46 of the leaflet can be detached from the inflow struts. The apex region 48 of leaflet can be located at the inflow end 616 of the frame 600. In the example depicted in FIG. 14, the apex region 48 of the leaflet is not connected to any strut of the frame 600. The apex 49 of the leaflet can be located upstream of a junction 625 where the axial support member 654 is connected to the inflow struts.

FIG. 15 shows a one-third section of an annular frame 700 for a prosthetic valve, according to another example. The frame 700 has four rows (for example rows 722, 724, 726, 728) of angled struts 702, wherein the first row 722 of angled struts define an outflow end 718 and the fourth row 728 of angled struts define an inflow end 716 of the frame 700. The upper half of the frame (closer to the outflow end 718) and the lower half of the frame (closer to the inflow end 716) mirror each other. For example, the frame 700 includes a plurality of first axial frame members 710 (similar to frame members 310) bridging the first row 722 and second row 724 of angled struts, as well as a plurality of second axial frame members 710′ (which mirror corresponding first axial frame members 710) bridging the third row 726 and fourth row 728 of angled struts. Additionally, the frame 700 includes a plurality of first support structures 750 and a plurality of second support structures 750′. Each first support structure 750 has two angled support arms 752 connecting two immediately adjacent first axial frame members 710 and one axial support member 754 bridging the two angled support arms 752 and the second row 724 of angle struts. Each second support structure 750′ has two angled support arms 752′ connecting two immediately adjacent second axial frame members 710′ and one axial support member 754′ bridging the two angled support arms 752′ and the third row 726 of angle struts. Thus, each second support structure 750′ defines an inverted Y-shaped configuration. The axial support members 754 of the first support structures 750 are connected to axial support members 754′ of the second support structures 750′ at junctions 760 between the second row 724 and third row 726 of angled struts such that each first support structure 750 mirrors a corresponding second support structure 750′.

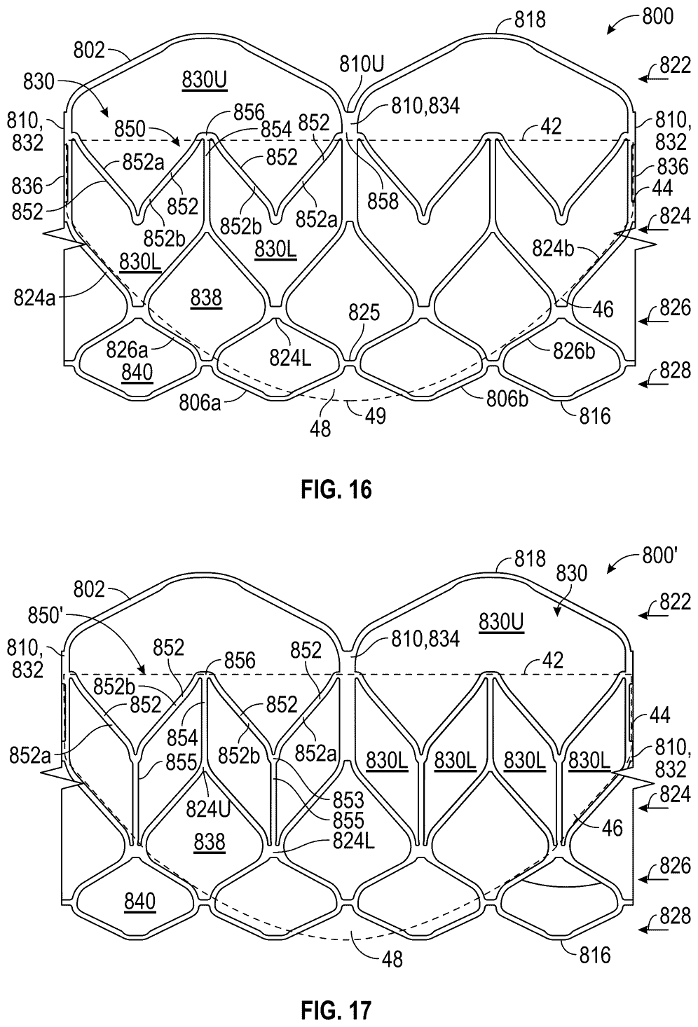

FIG. 16 shows a one-third section of an annular frame 800 for a prosthetic valve, according to another example. The frame 800 has four rows (for example rows 822, 824, 826, 828) of angled struts 802. The first row 822 of angled struts define an outflow end 818 and the fourth row 828 of angled struts define an inflow end 816 of the frame 800. Similarly, the frame 800 includes a plurality of axial frame members 810 (for example, three commissure supports 832 having respective commissure windows 836 and three axial posts 834) bridging the first row 822 and second row 824 of angled struts. The first and second rows 822, 824 of angled struts and the axial frame members 810 can define a row of eight-sided outflow cells 830. The second and third rows 824, 826 of angled struts can define an intermediate row of four-sided cells 838. The third and fourth rows 826, 828 of angled struts can define a row of four-sided inflow cells 840. In this example, there are six outflow cells 830, twelve inflow cells 840, and twelve cells 838 in the intermediate row.

The frame 800 also includes a plurality of support structures 850. As shown in FIG. 16, each support structure 850 can have four angled support arms 852 that are interconnected to define a W-shaped configuration. The four angled support arms 852 can include two side support arms 852a respectively connected to two adjacent axial frame members 810 and two middle support arms 852b positioned between the two side support arms 852a. The second row 824 of angled struts are joined together at alternating upper ends 824U and lower ends 824L. Each support structure 850 further comprises a middle axial support member 854 connecting a joint 856 of the two middle support arms 852b and a corresponding upper end 824U of the second row 824 of angled struts. As a result, each support structure 850 can divide a corresponding outflow cell 830 into an upper sub-cell 830U and two lower sub-cells 830L. As shown in FIG. 16, each lower sub-cell 830L is six-sided, and the upper sub-cell 830U has a width that is twice that of each lower sub-cell 830L. In the example depicted in FIG. 16, each side support arm 852a is connected to an adjacent axial frame member 810 at a connecting point 858 that is axially spaced away from an upper end 810U of the axial frame member 810. In other examples, the connecting point 858 can overlap with the upper end 810U.

FIG. 16 also illustrates relative positions of a leaflet (for example, 40 or 104) and the frame 800 after mounting the leaflet to the frame 800 (the outer perimeter of the leaflet is shown in dashed lines), according to one example. As shown, the outflow edge 42 of the leaflet can extend across two outflow cells 830. The axial position of the outflow edge 42 can be anywhere between upper ends of the commissure windows 836 and upper ends 810U of the axial frame member 810. Each commissure tab 44 of the leaflet can be paired with an adjacent commissure tab of an adjacent leaflet to form a commissure of a leaflet assembly that is inserted into a corresponding commissure window 836. The cusp edge 46 of the leaflet can extend along and be attached to at least four angled struts (for example, 824a, 824b, 826a, and 826b) respectively located on the second row 824 and third row 826. In some examples, the cusp edge 46 of the leaflet can also be attached to two angled strut portions (for example, 806a and 806b) of the inflow struts. Alternatively, the cusp edge 46 of the leaflet can be detached from the inflow struts. The apex region 48 of leaflet can be located at the inflow end 816 of the frame 800. In the example depicted in FIG. 16, the apex region 48 of the leaflet is not connected to any strut of the frame 800. The apex 49 of the leaflet can be located upstream of a junction 825 where the third row 826 of angled struts are connected to the fourth row 828 of angled struts.

FIG. 17 shows a one-third section of an annular frame 800′ for a prosthetic valve, according to another example. The frame 800′ is similar to the frame 800 except for having different support structures 850′. As shown in FIG. 17, each support structure 850′ also has four angled support arms 852 (including two side support arms 852a and two middle support arms 852b) that are interconnected to define a W-shaped configuration, and one middle axial support member 854 connecting the joint 856 of the two middle support members 852b and a corresponding upper end 824U of the second row 824 of angled struts. Additionally, each support structure 850′ further includes two side axial support members 855 located on opposite sides of the middle support member 854. Each side axial support member 855 can connect one of the lower ends 824L of the second row 824 of angled struts and a joint 853 formed between a side support arm 852a and an adjacent middle support arm 852b. As a result, each support structure 850′ can divide a corresponding outflow cell 830 into an upper sub-cell 830U and four lower sub-cells 830L. Each of the lower sub-cell 830L has a width that is one quarter of the width of the upper sub-cell 830U. As shown, each of the lower sub-cell 830L can have a parallelogram shape.

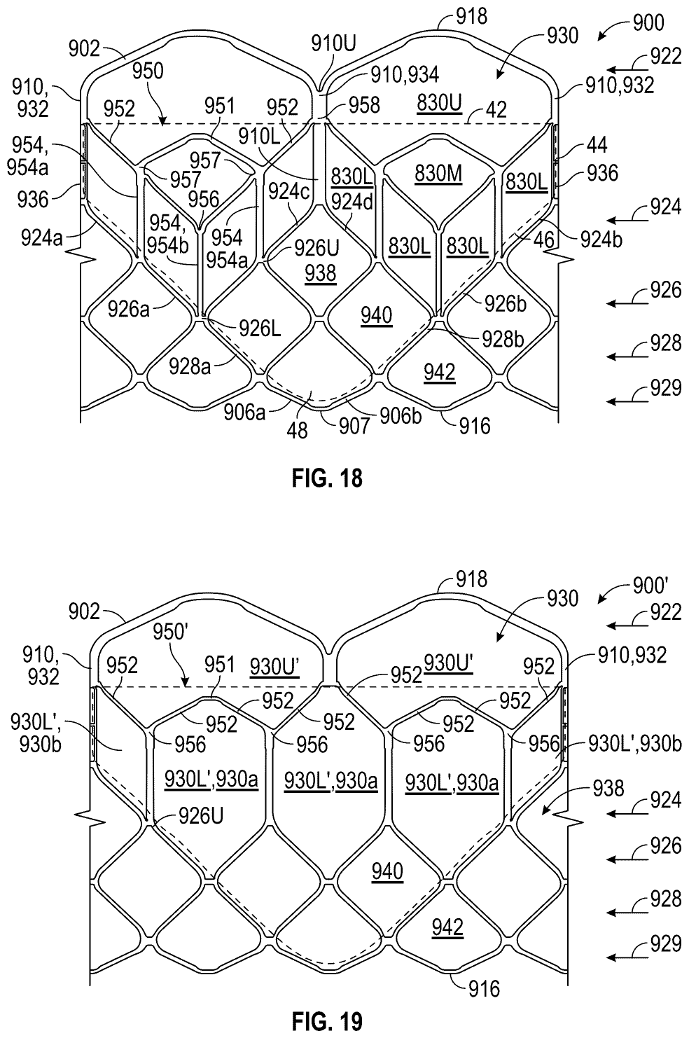

FIG. 18 shows a one-third section of an annular frame 900 for a prosthetic valve, according to another example. The frame 900 has five rows (for example rows 922, 924, 926, 928, 929) of angled struts 902. The first row 922 of angled struts define an outflow end 918 and the fifth row 929 of angled struts define an inflow end 916 of the frame 900. Similarly, the frame 900 includes a plurality of axial frame members 910 (for example, three commissure supports 932 having respective commissure windows 936 and three axial posts 934) bridging the first row 922 and second row 924 of angled struts.

In this example, while the angled struts in each of the other four rows (rows 922, 926, 928, 929) are interconnected to form a circumferentially continuous row of angled struts, the second row 924 of angled struts are discontinuous. For example, as shown in FIG. 18, two angled struts 924c, 924d in the second row 924 can be connected to a lower end 910L of a corresponding axial frame member 910. However, the same two angled struts 924c, 924d are not directly connected to any other angled struts (for example, 924a, 924b) in the second row 924 of angled struts. In this example, the second row 924 has only 12 angled struts, whereas each of the third row 926 and fourth row 928 has 24 angled struts.

In the example depicted in FIG. 18, the first, second, and third rows 922, 924, 926 of angled struts and the axial frame members 910 can define a row of six-sided outflow cells 930. The second and third rows 924, 926 of angled struts can define an upper intermediate row of four-sided cells 938. The third and fourth rows 826, 828 of angled struts can define a lower intermediate row of four-sided cells 940. The fourth and fifth rows 928, 928 of angled struts can define a row of four-sided inflow cells 942. In this example, there are six outflow cells 930, six cells 938 in the upper intermediate row, twelve cells 940 in the lower intermediate row, and twelve inflow cells 942. Due to the discontinuity of the angled struts in the second row 924, each cell 938 in the upper intermediate row is also not directly connected to any other cells 938 in the upper intermediate row.

The frame 900 also includes a plurality of support structures 950 connecting the plurality of axial frame members 910 and the second and third rows 924, 926 of angled struts. In this example, each support structure 950 comprises two angled support arms 952 connecting two immediately adjacent axial frame members 910 and three axial support members 954 bridging the two angled support arms 952 and the second and third rows 924, 926 of angle struts. As shown in FIG. 18, the three axial support members 954 include one middle axial support member 954b and two side axial support members 954a. The middle axial support member 954b has an upper end connected to a joint 956 of the two angled support arms 952. The two side axial support members 954a have respective upper ends connected to corresponding mid-points 957 of the two angled support arms 952 (that is, each side axial support member 954a bisects a corresponding angled support arm 952). Additionally, each support structure 950 further includes one connecting support member 951 which connects the mid-points 957 of the two angled support arms 952. For a prosthetic valve having an outer skirt, the outer skirt can be attached to at least parts of the support structures 950 (for example, parts of the angled support arms 952 and/or the connecting support member 951).

In some examples, the angled support arms 952 and the connecting support member 951 are narrower than the outflow struts (in the first row 922) and the inflow struts (in the fifth row 929). In some examples, the axial support members 954 are narrower than the axial frame members 910.

As shown in FIG. 18, two angled support arms 952 of two adjacent support structures 950 located on opposite sides of an axial frame member 910 can be connected to the axial frame member 910 at a connecting point 958. In the depicted example, the connecting point 958 is axially spaced away from an upper end 910U of the axial frame member 910. In other examples, the upper end 910U of the axial frame member 910 can overlap with the connecting point 958.

The third row 926 of angled struts are joined together at alternating upper ends 926U and lower ends 926L. Each angled strut in the second row 924 of angled struts is connected to one of the upper ends 926U of the third row 926 of angled struts. As depicted in FIG. 18, for each support structure 950, a lower end of the middle axial support member 954b can be connected to one of the lower ends 926L of the third row 926 of angled struts, and a lower end of each side axial support member 954a can be connected to one of the upper end 926U of the third row 926 of angled struts.

In the example depicted in FIG. 18, each support structure 950 can divide a corresponding outflow cell 930 into six sub-cells, including a row of four lower sub-cells 930L arranged in a V-shaped pattern, one middle sub-cell 930M, and one upper sub-cell 930U. Each of four lower sub-cells 930L has a parallelogram shape. The middle sub-cell 930M has four sides. The upper sub-cell 930U can have eight sides (if the connecting point 958 is axially spaced away from the upper end 910U of the axial frame member 910) or have six sides (if the upper end 910U of the axial frame member 910 overlaps with the connecting point 958).

FIG. 18 also illustrates relative positions of a leaflet (for example, 40 or 104) and the frame 900 after mounting the leaflet to the frame 900 (the outer perimeter of the leaflet is shown in dashed lines), according to one example. As shown, the outflow edge 42 of the leaflet can extend across two outflow cells 930. The axial position of the outflow edge 42 can be anywhere between upper ends of the commissure windows 936 and upper ends 910U of the axial frame member 910. Each commissure tab 44 of the leaflet can be paired with an adjacent commissure tab of an adjacent leaflet to form a commissure of a leaflet assembly that is inserted into a corresponding commissure window 936. The cusp edge 46 of the leaflet can extend along and be attached to six angled struts (for example, struts 924a, 924b, 926a, 926b, 928a, and 928b) respectively located in the second row 924, third row 926, and fourth row 928, and two angled strut portions (for example, struts 906a and 906b) located on the fifth row 929. The apex region 48 of leaflet can align with a curved apex portion 907 (similar to 207 of FIGS. 5A-5B) of an inflow strut at the inflow end 916 of the frame 900.

FIG. 19 shows a one-third section of an annular frame 900′ for a prosthetic valve, according to another example. Similar to the frame 900, the frame 900′ has five rows (for example 922, 924, 926, 928, 929) of angled struts 902, in which the first row 922 of angled struts define an outflow end 918 and the fifth row 929 of angled struts define an inflow end 916 of the frame 900. The frame 900′ also includes a plurality of axial frame members 910 bridging the first row 922 and second row 924 of angled struts. Unlike the frame 900, the axial frame members 910 of the frame 900′ includes three commissure supports 932 but no axial posts (for example, posts 934) located between the commissure supports 932 and bridging the first and second rows 922, 924 of angled struts. Similar to the frame 900, while the angled struts in each of the other four rows (922, 926, 928, 929) are interconnected to form a circumferentially continuous row of angled struts, the second row 924 of angled struts are discontinuous. In this example, the second row 924 has only 6 angled struts, whereas each of the third row 926 and fourth row 928 has 24 angled struts.

In the example depicted in FIG. 19, the first, second, and third rows 922, 924, 926 of angled struts and the axial frame members 910 can define a row of 12-sided outflow cells 930. The second and third rows 924, 926 of angled struts can define an upper intermediate row of four-sided cells 938 (located directly below the corresponding commissure supports 932). The third and fourth rows 826, 828 of angled struts can define a lower intermediate row of four-sided cells 940. The fourth and fifth rows 928, 928 of angled struts can define a row of four-sided inflow cells 942. In this example, due to the missing of axial posts, there are only three outflow cells 930 and three cells 938 in the upper intermediate row, while there are twelve cells 940 in the lower intermediate row, and twelve inflow cells 942. Due to the discontinuity of the angled struts in the second row 924, the cells 938 in the upper intermediate row are not directly connected to one another.

The frame 900′ can have three support structures 950′ connecting the three axial frame members 910 and the second and third rows 924, 926 of angled struts. As shown in FIG. 19, each support structure 950′ includes eight angled support arms 952 and four axial support members 954. Each axial support member 954 has an upper end connected to a corresponding joint 956 of two angled support arms 952 and a lower end connected to a corresponding upper end 926U of the third row 926 of angled struts. In this example, each support structure 950 can divide a corresponding outflow cell 930 seven sub-cells, including a row of five lower sub-cells 930L′ and two upper sub-cells 930U′. The five lower sub-cells 930L′ include three six-sided sub-cells 930a located between two four-sided sub-cells 930b (each having a parallelogram shape).

### Exemplary Expansion Mechanisms of Prosthetic Valves

For any of the prosthetic valves described herein, the frame can be made of any of various suitable plastically-expandable materials. When constructed of a plastically-expandable material, the frame (and thus the prosthetic valve) can be crimped to a radially collapsed configuration on a delivery catheter or apparatus and then expanded inside a patient by an inflatable balloon or equivalent expansion mechanism. In certain examples, during delivery to the implantation site, the prosthetic valve can be placed inside of a delivery capsule or sheath to protect against the prosthetic valve contacting the patient's vasculature, such as when the prosthetic valve is advanced through a femoral artery. The capsule can also retain the prosthetic valve in a radially compressed state having a slightly smaller diameter and crimp profile than may be otherwise possible without a capsule by preventing any recoil (expansion) of the frame once it is crimped onto the delivery apparatus.

Suitable plastically-expandable materials that can be used to form any of the frames disclosed herein include, metal alloys, polymers, or combinations thereof. Example metal alloys can comprise one or more of the following: nickel, cobalt, chromium, molybdenum, titanium, or other biocompatible metal. In some examples, the frame can comprise stainless steel. In some examples, the frame can comprise cobalt-chromium. In some examples, the frame can comprise nickel-cobalt-chromium. In some examples, the frame comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details on balloon expandable prosthetic valves can be found in U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein.

Any of the prosthetic valves described herein can be self-expandable. For example, the frame of the prosthetic valve can comprise a shape-memory material (for example, Nitinol). When the prosthetic valve is self-expandable, the frame (and thus the prosthetic valve) can be crimped to a radially compressed configuration and restrained in the compressed configuration by insertion into a sheath or equivalent mechanism of a delivery catheter. Once inside the body at the desired implantation site, the prosthetic valve can be deployed or released from the delivery sheath, which allows the prosthetic valve to expand to its functional size. In some examples, the frame (and therefore the prosthetic valve) can partially self-expand from the radially compressed configuration to a partially radially expanded configuration. The frame (and therefore the prosthetic valve) can be further radially expanded from the partially expanded configuration to a further radially expanded configuration via one or more actuation assemblies (for example, an inflatable balloon and/or one or more mechanical actuators) of the delivery apparatus.

Additional details regarding exemplary self-expandable prosthetic valves and the related delivery apparatus/catheters/systems are described in U.S. Pat. Nos. 8,652,202, 9,155,619, and 9,867,700, all of which are incorporated herein by reference.

Additionally, and/or alternatively, any of the prosthetic valves described herein can be mechanically expandable. For example, the struts of the frame can be pivotably coupled to one another at one or more pivot joints along the length of each strut. As a result, an axial force applied to the frame (for example, pressing the inflow end and the outflow end of the frame toward each other or pulling the inflow end and the outflow end of the frame away from each other) can cause the prosthetic valve to radially expand or compress. The axial force can be generated by actuating one or more mechanical actuators of the delivery apparatus that are operatively coupled to the frame.

Additional details regarding exemplary mechanically-expandable prosthetic valves and the related delivery apparatus/catheters/systems are described in U.S. Patent Application Publication Nos. 2018/0153689, 2018/0311039, 2019/0060057, and PCT Patent Application Publication No. WO/2021/188476, all of which are incorporated by reference herein.

### Exemplary Delivery Apparatus

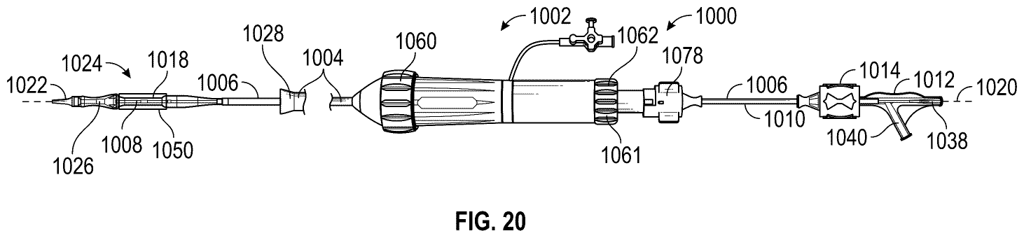

FIG. 20 shows a delivery apparatus 1000, according to an example, that can be used to implant an expandable prosthetic valve (for example, the prosthetic valve 10, 100, 150 and/or any of the other prosthetic valves described herein). In some examples, the delivery apparatus 300 can be specifically adapted for use in introducing a prosthetic valve into a heart.

The delivery apparatus 1000 in the illustrated example of FIG. 20 is a balloon catheter comprising a handle 1002 and a steerable, outer shaft 1004 extending distally from the handle 1002. The delivery apparatus 1000 can further comprise an intermediate shaft 1006 (which also may be referred to as a balloon shaft) that extends proximally from the handle 1002 and distally from the handle 1002, the portion extending distally from the handle 1002 also extending coaxially through the outer shaft 1004. Additionally, the delivery apparatus 1000 can further comprise an inner shaft 1008 extending distally from the handle 1002 coaxially through the intermediate shaft 1006 and the outer shaft 1004 and proximally from the handle 1002 coaxially through the intermediate shaft 1006.

The outer shaft 1004 and the intermediate shaft 1006 can be configured to translate (for example, move) longitudinally, along a central longitudinal axis 1020 of the delivery apparatus 1000, relative to one another to facilitate delivery and positioning of a prosthetic valve at an implantation site in a patient's body.

The intermediate shaft 1006 can include a proximal end portion 1010 that extends proximally from a proximal end of the handle 1002, to an adaptor 1012. A rotatable knob 1014 can be mounted on the proximal end portion 1010 and can be configured to rotate the intermediate shaft 1006 around the central longitudinal axis 1020 and relative to the outer shaft 1004.

The adaptor 1012 can include a first port 1038 configured to receive a guidewire therethrough and a second port 1040 configured to receive fluid (for example, inflation fluid) from a fluid source. The second port 1040 can be fluidly coupled to an inner lumen of the intermediate shaft 1006.

The intermediate shaft 1006 can further include a distal end portion that extends distally beyond a distal end of the outer shaft 1004 when a distal end of the outer shaft 1004 is positioned away from an inflatable balloon 1018 of the delivery apparatus 1000. A distal end portion of the inner shaft 1008 can extend distally beyond the distal end portion of the intermediate shaft 1006.

The balloon 1018 can be coupled to the distal end portion of the intermediate shaft 1006.

In some examples, a distal end of the balloon 1018 can be coupled to a distal end of the delivery apparatus 1000, such as to a nose cone 1022, or to an alternate component at the distal end of the delivery apparatus 1000 (for example, a distal shoulder). An intermediate portion of the balloon 1018 can overlay a valve mounting portion 1024 of a distal end portion of the delivery apparatus 1000 and a distal end portion of the balloon 1018 can overly a distal shoulder 1026 of the delivery apparatus 1000. The valve mounting portion 1024 and the intermediate portion of the balloon 1018 can be configured to receive a prosthetic valve in a radially compressed state. For example, as shown schematically in FIG. 20, a prosthetic valve 1050 (which can be one of the prosthetic valves described herein) can be mounted around the balloon 1018, at the valve mounting portion 1024 of the delivery apparatus 1000.

The balloon shoulder assembly, including the distal shoulder 1026, can be configured to maintain the prosthetic valve 1050 (or other medical device) at a fixed position on the balloon 1018 during delivery through the patient's vasculature.

The outer shaft 1004 can include a distal tip portion 1028 mounted on its distal end. The outer shaft 1004 and the intermediate shaft 1006 can be translated axially relative to one another to position the distal tip portion 1028 adjacent to a proximal end of the valve mounting portion 1024, when the prosthetic valve 1050 is mounted in the radially compressed state on the valve mounting portion 1024 (as shown in FIG. 20) and during delivery of the prosthetic valve to the target implantation site. As such, the distal tip portion 1028 can be configured to resist movement of the prosthetic valve 1050 relative to the balloon 1018 proximally, in the axial direction, relative to the balloon 1018, when the distal tip portion 1028 is arranged adjacent to a proximal side of the valve mounting portion 1024.

An annular space can be defined between an outer surface of the inner shaft 1008 and an inner surface of the intermediate shaft 1006 and can be configured to receive fluid from a fluid source via the second port 1040 of the adaptor 1012. The annular space can be fluidly coupled to a fluid passageway formed between the outer surface of the distal end portion of the inner shaft 1008 and an inner surface of the balloon 1018. As such, fluid from the fluid source can flow to the fluid passageway from the annular space to inflate the balloon 1018 and radially expand and deploy the prosthetic valve 1050.

An inner lumen of the inner shaft can be configured to receive a guidewire therethrough, for navigating the distal end portion of the delivery apparatus 1000 to the target implantation site.

The handle 1002 can include a steering mechanism configured to adjust the curvature of the distal end portion of the delivery apparatus 1000. In the illustrated example, for example, the handle 1002 includes an adjustment member, such as the illustrated rotatable knob 1060, which in turn can be operatively coupled to the proximal end portion of a pull wire. The pull wire can extend distally from the handle 1002 through the outer shaft 1004 and has a distal end portion affixed to the outer shaft 1004 at or near the distal end of the outer shaft 1004. Rotating the knob 1060 can increase or decrease the tension in the pull wire, thereby adjusting the curvature of the distal end portion of the delivery apparatus 1000. Further details on steering or flex mechanisms for the delivery apparatus can be found in U.S. Pat. No. 9,339,384, which is incorporated by reference herein.

The handle 1002 can further include an adjustment mechanism 1061 including an adjustment member, such as the illustrated rotatable knob 1062, and an associated locking mechanism including another adjustment member, configured as a rotatable knob 1078. The adjustment mechanism 1061 can be configured to adjust the axial position of the intermediate shaft 1006 relative to the outer shaft 1004 (for example, for fine positioning at the implantation site). Further details on the delivery apparatus 1000 can be found in PCT Application No. PCT/US2021/047056, which is incorporated by reference herein.

Although the delivery apparatus 1000 depicted in FIG. 20 is specifically adapted to deliver a balloon expandable prosthetic valve, it is to be understood that variants of the delivery apparatus 1000 can be adapted for delivery of self-expandable prosthetic valves and/or mechanically expandable prosthetic valves, as described in references incorporated above.

### Exemplary Delivery Techniques

For implanting a prosthetic valve within the native aortic valve via a transfemoral delivery approach, the prosthetic valve can be mounted in a radially compressed state along the distal end portion of a delivery apparatus. The prosthetic valve and the distal end portion of the delivery apparatus can be inserted into a femoral artery and then advanced into and through the descending aorta, around the aortic arch, and through the ascending aorta. The prosthetic valve can be positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) can be introduced into the left ventricle through a surgical opening in the chest and the apex of the heart and the prosthetic valve can be positioned within the native aortic valve. Alternatively, in a transaortic procedure, a prosthetic valve (on the distal end portion of the delivery apparatus) can be introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, into the right atrium, across the atrial septum (for example, through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) can be introduced into the left ventricle through a surgical opening in the chest and the apex of the heart and the prosthetic valve can be positioned within the native mitral valve.

For implanting a prosthetic valve within the native tricuspid valve, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, and into the right atrium, and the prosthetic valve can be positioned within the native tricuspid valve. A similar approach can be used for implanting the prosthetic valve within the native pulmonary valve or the pulmonary artery, except that the prosthetic valve can be advanced through the native tricuspid valve into the right ventricle and toward the pulmonary valve/pulmonary artery.

Another delivery approach is a trans-atrial approach whereby a prosthetic valve (on the distal end portion of the delivery apparatus) can be inserted through an incision in the chest and an incision made through an atrial wall (of the right or left atrium) for accessing any of the native heart valves. Atrial delivery can also be made intravascularly, such as from a pulmonary vein. Still another delivery approach is a trans-ventricular approach whereby a prosthetic valve (on the distal end portion of the delivery apparatus) can be inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

It is to be understood that the treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising: an annular frame that is radially collapsible to a collapsed configuration and radially expandable to an expanded configuration; and an outer skirt disposed on an outer surface of the annular frame, wherein the annular frame comprises: an inflow end; an outflow end; a first row of angled struts defining the outflow end; a second row of angled struts being closer to the inflow end than the first row of angled struts; a plurality of axial frame members bridging the first row of angled struts and the second row of angled struts; and a plurality of support members connecting the plurality of axial frame members, wherein the support members are narrower than the axial frame members and the first and second rows of angled struts, wherein the outer skirt is connected to the support members.

Example 2. The prosthetic valve of any example herein, particular example 1, further comprising a leaflet structure positioned within the annular frame and configured to permit blood flow from the inflow end to the outflow end and block blood fluid flow from the outflow end to the inflow end.

Example 3. The prosthetic valve of any example herein, particular example 2, further comprising an inner skirt attached to an inner surface of the annular frame, wherein the leaflet structure is connected to the inner skirt.

Example 4. The prosthetic valve of any example herein, particularly any one of examples 2-3, wherein the plurality of axial frame members comprises a plurality of axially extending commissure supports and one or more axial posts located between any two immediately adjacent commissure supports, wherein each commissure support is configured to support a corresponding commissure of the leaflet structure.

Example 5. The prosthetic valve of any example herein, particular example 4, wherein the leaflet structure comprises three leaflets defining three commissures, and the plurality of axial frame members comprise three axially extending commissure supports.

Example 6. The prosthetic valve of any example herein, particularly any one of examples 4-5, wherein there are two axial posts located between any two immediately adjacent commissure supports.

Example 7. The prosthetic valve of any example herein, particularly any one of examples 1-6, wherein the first and second rows of angled struts are narrower than the plurality of axial frame members.

Example 8. The prosthetic valve of any example herein, particularly any one of examples 1-7, wherein the second row of angled struts are narrower than the first row of angled struts.

Example 9. The prosthetic valve of any example herein, particularly any one of examples 1-8, wherein the first row of angled struts are joined together at a first set of alternating upper ends and lower ends, the upper ends defining the outflow end and the lower ends being closer to the inflow end than the upper ends, wherein the second row of angled struts are joined together at a second set of alternating upper ends and lower ends, the upper ends being closer to the outflow end than the lower ends, wherein the plurality of axial frame members respectively connect the lower ends of the first row of angled struts to the upper ends of the second row of angled struts.

Example 10. The prosthetic valve of any example herein, particular example 9, wherein each support member is connected to the upper ends of two immediately adjacent axial frame members.

Example 11. The prosthetic valve of any example herein, particular example 9, wherein each support member is connected to two immediately adjacent axial frame members at respective connecting points, wherein the connecting points are located between the lower ends of the first row and the upper ends of the second row connected by the respective axial frame members.

Example 12. The prosthetic valve of any example herein, particular example 11, wherein at least some of the support members comprise openings that are axially spaced away from the corresponding connecting points.

Example 13. The prosthetic valve of any example herein, particularly any one of examples 1-12, wherein the annular frame comprises at least four rows of angled struts.

Example 14. The prosthetic valve of any example herein, particular example 13, wherein the annular frame further comprises a third row of angled struts being closer to the inflow end than the second row of angled struts and a fourth row of angled struts defining the inflow end.

Example 15. The prosthetic valve of any example herein, particular example 14, wherein the third row of angled struts are narrower than the fourth row of angled struts.

Example 16. The prosthetic valve of any example herein, particularly any one of examples 14-15, wherein the second row of angled struts have the same width as the third row of angled struts.

Example 17. The prosthetic valve of any example herein, particularly any one of examples 14-16, wherein the fourth row of angled struts have the same width as the first row of angled struts.

Example 18. The prosthetic valve of any example herein, particularly any one of examples 14-17, wherein the first row of angled struts, the second row of angled struts, and the plurality of axial frame members define a plurality of first cells of the annular frame, wherein the support members divide the first cells into respective upper cell portions and lower cell portions, the upper cell portions being closer to the outflow end than the lower cell portions.

Example 19. The prosthetic valve of any example herein, particular example 18, wherein the first cells have a hexagonal shape when the annular frame is in the expanded configuration.

Example 20. The prosthetic valve of any example herein, particularly any one of examples 18-19, wherein the upper cell portions are larger than the lower cell portions when the annular frame is in the expanded configuration.

Example 21. The prosthetic valve of any example herein, particularly any one of examples 18-19, wherein the lower cell portions are larger than the upper cell portions when the annular frame is in the expanded configuration.

Example 22. The prosthetic valve of any example herein, particularly any one of examples 18-19, wherein the upper cell portions and the lower cell portions have the same size when the annular frame is in the expanded configuration.

Example 23. The prosthetic valve of any example herein, particularly any one of examples 18-22, wherein the lower cell portions have a quadrilateral shape when the annular frame is in the expanded configuration.

Example 24. The prosthetic valve of any example herein, particularly any one of examples 18-22, wherein none of the upper and lower portions have a quadrilateral shape when the annular frame is in the expanded configuration.

Example 25. The prosthetic valve of any example herein, particularly any one of examples 18-24, wherein the second row of angled struts and the third row of angled struts define a plurality of second cells of the annular frame, wherein the third row of angled struts and the fourth row of angled struts define a plurality of third cells of the annular frame.

Example 26. The prosthetic valve of any example herein, particular example 25, wherein each of the second cells and the third cells has a quadrilateral shape when the annular frame is in the expanded configuration.

Example 27. The prosthetic valve of any example herein, particularly any one of examples 22-26, wherein the second cells and the third cells have different shapes when the annular frame is in the expanded configuration.

Example 28. The prosthetic valve of any example herein, particularly any one of examples 1-27, wherein each support member comprises two arm portions and a joint portion connecting the two arm portions, wherein each support member is configured to fold at the joint portion when the annular frame moves from the expanded configuration to the collapsed configuration.

Example 29. The prosthetic valve of any example herein, particular example 28, wherein the joint portion of each support member is configured to move axially toward the inflow end when the annular frame moves from the expanded configuration to the collapsed configuration.

Example 30. The prosthetic valve of any example herein, particular example 28, wherein the joint portion of each support member is configured to move axially toward the outflow end when the annular frame moves from the expanded configuration to the collapsed configuration.

Example 31. A prosthetic valve, comprising: an annular frame that is radially expandable and compressible; and an outer skirt disposed on an outer surface of the annular frame, wherein the annular frame comprises: an inflow end; an outflow end; a first row of angled struts defining the outflow end and a second row of angled struts upstream of the first row of angled struts; a plurality of axial frame members comprising respective first ends and second ends, the first ends being connected to the first row of angled struts and the second ends being connected to the second row of angled struts; and a plurality of support members connecting the plurality of axial frame members at connecting points located between the first ends and the second ends, wherein the outer skirt is connected to the support members.

Example 32. The prosthetic valve of any example herein, particular example 31, further comprising a plurality of leaflets positioned within the annular frame and configured to permit blood flow from the inflow end to the outflow end and block blood fluid flow from the outflow end to the inflow end.

Example 33. The prosthetic valve of any example herein, particular example 32, wherein the plurality of axial frame members comprises a plurality of axially extending commissure supports and one or more axial posts located between any two immediately adjacent commissure supports, wherein each commissure support comprises a commissure window configured to receive a corresponding commissure of two adjacent leaflets.

Example 34. The prosthetic valve of any example herein, particular example 33, wherein the plurality of axial frame members comprise three axially extending commissure supports and exactly two axial posts located between any two immediately adjacent commissure supports.

Example 35. The prosthetic valve of any example herein, particularly any one of examples 33-34, wherein at least some of the support members connect to the commissure supports at the connecting points located between the first ends and the commissure windows.

Example 36. The prosthetic valve of any example herein, particularly any one of examples 33-34, wherein at least some of the support members connect to the commissure supports at the connecting points located between the second ends and the commissure windows.

Example 37. The prosthetic valve of any example herein, particularly any one of examples 31-36, wherein the support members are narrower than the axial frame members and the first and second rows of angled struts.

Example 38. The prosthetic valve of any example herein, particularly any one of examples 31-37, wherein the annular frame further comprises a third row of angled struts upstream of the second rows of angled struts and a fourth rows of angled struts upstream of the third rows of angled struts, wherein the fourth row of angled struts defines the inflow end.

Example 39. The prosthetic valve of any example herein, particular example 38, wherein second row of angled struts and the third row of angled struts have the same width, and the first row of angled struts and the fourth row of angled struts have the same width.

Example 40. The prosthetic valve of any example herein, particularly any one of examples 38-39, wherein the axial frame members are wider than any of the first, second, third, and fourth rows of angled struts.

Example 41. An annular frame that is radially expandable and compressible, the annular frame comprising: an inflow end; an outflow end; a row of outflow struts defining the outflow end; a row of inflow struts defining the inflow end; an intermediate row of struts located axially between the row of outflow struts and the row of inflow struts; a plurality of axial frame members comprising respective first ends and second ends, the first ends being connected to the row of outflow struts and the second ends being connected to the intermediate row of struts; and a circumferentially extending row of support members located axially between the row of outflow struts and the intermediate row of struts, the support members connecting the plurality of axial frame members, wherein each outflow strut comprises two angled strut portions interconnected by an apex portion, wherein the support members are narrower than the axial frame members, the angled strut portions of the outflow struts, and the struts of the intermediate row of struts.

Example 42. The annular frame of any example herein, particular example 41, wherein the apex portions are narrower than the angled strut portions of the corresponding outflow struts.

Example 43. The annular frame of any example herein, particularly any one of examples 41-42, wherein each apex portion curves between a pair of two angled strut portions of a corresponding outflow strut.

Example 44. The annular frame of any example herein, particularly any one of examples 41-43, wherein each apex portion has an arc length that extends along at least 25% of a total arc length of a corresponding outflow strut.

Example 45. The annular frame of any example herein, particular example 41, wherein each apex portion forms a U-shaped bend between the two angled strut portions of a corresponding outflow strut.

Example 46. The annular frame of any example herein, particularly any one of examples 41-45, wherein each support member comprises two arm portions and a joint portion connecting the two arm portions, wherein the two arm portions forms an obtuse angle at the joint portion when the annular frame is radially expanded.

Example 47. The annular frame of any example herein, particular example 46, wherein the joint portion forms a U-shaped bend between the two arm portions.

Example 48. The annular frame of any example herein, particularly any one of examples 46-47, wherein the joint portion of each support member is configured to move axially toward the inflow end when the annular frame is radially compressed and moved axially toward the outflow end when the annular frame is radially expanded.

Example 49. The annular frame of any example herein, particularly any one of examples 46-47, wherein the joint portion of each support member is configured to move axially toward the outflow end when the annular frame is radially compressed and moved axially toward the inflow end when the annular frame is radially expanded.

Example 50. The annular frame of any example herein, particularly any one of examples 46-49, wherein each arm portion is substantially straight.

Example 51. The annular frame of any example herein, particularly any one of examples 46-50, wherein each arm portion is connected to a corresponding axial frame member via a curved attachment portion.

Example 52. The annular frame of any example herein, particularly any one of examples 46-51, wherein each support member is configured to fold at the joint portion when the annular frame is radially compressed so that the two arm portions extend substantially parallel to and between two immediately adjacent axial frame members.

Example 53. The annular frame of any example herein, particularly any one of examples 46-52, wherein the joint portions are closer to the outflow end than the second ends of the axial frame members when the annular frame is radially expanded.

Example 54. The annular frame of any example herein, particular example 53, wherein the joint portions are axially aligned with a midpoint between the first ends and the second ends when the annular frame is radially expanded.

Example 55. The annular frame of any example herein, particularly any one of examples 41-54, wherein a width of the support members ranges between 0.1 mm and 0.3 mm.

Example 56. The annular frame of any example herein, particular example 55, wherein the width of the support member ranges between 0.18 mm and 0.22 mm.

Example 57. The annular frame of any example herein, particularly any one of examples 41-56, wherein a width of the angled strut portions of the outflow struts ranges between 0.3 mm and 0.5 mm.

Example 58. The annular frame of any example herein, particular example 57, wherein the width the angled strut portions of the outflow struts ranges between 0.38 mm and 0.42 mm.

Example 59. The annular frame of any example herein, particularly any one of examples 41-58, wherein a width of the struts of the intermediate row of struts ranges between 0.15 mm and 0.35 mm.

Example 60. The annular frame of any example herein, particular example 59, wherein the width of the struts of the intermediate row of struts ranges between 0.23 mm and 0.27 mm.

Example 61. The annular frame of any example herein, particularly any one of examples 41-60, wherein a width of the axial frame members ranges between 0.2 mm and 2 mm.

Example 62. The annular frame of any example herein, particular example 61, wherein the width of the axial frame members ranges between 0.8 mm and 1.2 mm.

Example 63. The annular frame of any example herein, particularly any one of examples 41-62, wherein a width of the apex portions of the outflow struts ranges between 0.15 mm and 0.25 mm.

Example 64. The annular frame of any example herein, particular example 63, wherein the width of the apex portions of the outflow struts ranges between 0.18 mm and 0.22 mm.

Example 65. The annular frame of any example herein, particularly any one of examples 41-64, wherein the intermediate row of struts is a first intermediate row of struts, wherein there are one or more additional intermediate rows of struts located axially between the first intermediate row of struts and the row of inflow struts.

Example 66. The annular frame of any example herein, particular example 65, wherein there are exactly two intermediate rows of struts located axially between the row of outflow struts and the row of inflow struts.

Example 67. The annular frame of any example herein, particularly any one of examples 41-66, wherein the plurality of support members are connected to the plurality of axial frame members at connecting points located between the first ends and the second ends.

Example 68. The annular frame of any example herein, particular example 67, wherein at least some of the axial frame members comprise openings.

Example 69. The annular frame of any example herein, particular example 68, wherein the openings are axially spaced apart from the corresponding connecting points.

Example 70. The annular frame of any example herein, particularly any one of examples 68-69, wherein the axial frame members with the openings are wider than the axial frame members without the openings.

Example 71. An annular frame that is radially expandable and compressible, the annular frame comprising: a row of first end struts defining a first terminal end of the annular frame; a row of second end struts defining a second terminal end of the annular frame; a plurality of interconnected struts arranged in one or more rows circumferentially extending between the row of first end struts and the row of second end struts; a plurality of axial frame members comprising respective first ends and second ends, the first ends being connected to the row of first end struts and the second ends being connected to a first row of interconnected struts; and a plurality of support members connecting the plurality of axial frame members at connecting points that are axially located between the first ends and the second ends.

Example 72. The annular frame of any example herein, particular example 71, wherein connecting points are axially closer to the first ends than a midpoint between the first ends and the second ends.

Example 73. The annular frame of any example herein, particular example 71, wherein a midpoint between the first ends and the second ends is axially closer to the first ends than the connecting points.

Example 74. The annular frame of any example herein, particularly any one of examples 71-73, wherein each support member comprises two angled arm portions and a joint portion connecting the two angled arm portions.

Example 75. The annular frame of any example herein, particular example 74, wherein the joint portions of the support members are axially closer to the second ends than the connecting points when the annular frame is radially expanded.

Example 76. The annular frame of any example herein, particular example 74, wherein the joint portions of the support members are axially closer to the first ends than the connecting points when the annular frame is radially expanded.

Example 77. The annular frame of any example herein, particularly any one of examples 74-76, wherein the joint portions of the support members are axially located between the first ends and the second ends of the axial frame members when the annular frame is radially expanded.

Example 78. The annular frame of any example herein, particularly any one of examples 74-76, wherein the joint portions are axially aligned with the first ends of the axial frame members when the annular frame is radially expanded.

Example 79. The annular frame of any example herein, particularly any one of examples 74-78, wherein an axial distance between the second terminal end and the joint portions is at least 10 mm when the annular frame is radially expanded.

Example 80. The annular frame of any example herein, particular example 79, wherein the axial distance between the second terminal end and the joint portions is between one third and two thirds of a height of the annular frame when the annular frame is radially expanded, wherein the height of the annular frame is measured between the first terminal end and the second terminal end.

Example 81. The annular frame of any example herein, particularly any one of examples 71-80, wherein each first end strut comprises two angled strut portions interconnected by an apex portion.

Example 82. The annular frame of any example herein, particular example 81, wherein the support members are narrower than the axial frame members and the angled strut portions of the first end struts.

Example 83. The annular frame of any example herein, particularly any one of examples 71-92, wherein the support members comprise a fully annealed metal.

Example 84. The annular frame of any example herein, particularly any one of examples 71-83, wherein there is exactly one circumferentially extending row of interconnected struts linking the second ends of the plurality of axial frame members.

Example 85. An assembly, comprising: a prosthetic device comprising a frame, the prosthetic device being movable between a radially expanded state and a radially compressed state; and a delivery apparatus configured to deliver the prosthetic device in the radially compressed state to a target location; wherein the frame comprises: a row of outflow struts defining an outflow end of the frame; a row of inflow struts defining an inflow end of the frame; a row of interconnected struts circumferentially extending between the row of outflow struts and the row of inflow struts; a plurality of axial frame members comprising respective first ends and second ends, the first ends being connected to the row of outflow struts and the second ends being connected to the row of interconnected struts; and a plurality of support members connecting the plurality of axial frame members and extending between the row of outflow struts and the row of interconnected struts, wherein each outflow strut comprises two angled strut portions interconnected by an apex portion, wherein the support members are narrower than the axial frame members, the angled strut portions of outflow struts, and the row of interconnected struts.

Example 86. The assembly of any example herein, particular example 85, wherein the plurality of support members are connected to the plurality of axial frame members at connecting points that are axially located between the first ends and the second ends.

Example 87. The assembly of any example herein, particularly any one of examples 85-86, wherein the prosthetic device further comprises a leaflet structure positioned within the frame and configured to permit blood flow from the inflow end to the outflow end and block blood fluid flow from the outflow end to the inflow end.

Example 88. The assembly of any example herein, particular example 87, wherein the plurality of axial frame members comprises a plurality of commissure supports and one or more axial posts located between any two immediately adjacent commissure supports, wherein each commissure support is configured to support a corresponding commissure of the leaflet structure.

Example 89. The assembly of any example herein, particularly any one of examples 85-88, wherein the prosthetic device further comprises an outer skirt disposed on an outer surface of the frame, wherein the outer skirt is connected to the support members.

Example 90. The assembly of any example herein, particular example 89, wherein the outer skirt extends from the inflow end of the frame to an outflow edge of the outer skirt, wherein the outflow edge of the outer skirt is axially closer to the outflow end of the frame than the second ends of the axial frame members.

Example 91. The assembly of any example herein, particular example 90, wherein the outflow edge of the outer skirt is axially located between the first ends and the second ends of the axial frame members.

Example 92. The assembly of any example herein, particular example 90, wherein the outflow edge of the outer skirt is axially aligned with the first ends of the axial frame members.

Example 93. The assembly of any example herein, particularly any one of examples 85-92, wherein the delivery apparatus comprises a balloon shaft and a balloon mounted on a distal end portion of the balloon shaft, wherein the prosthetic device is crimped on the balloon when delivery the prosthetic device to the target location.

Example 94. The assembly of any example herein, particularly any one of examples 85-92, wherein the prosthetic device is self-expandable from the radially compressed state to the radially expanded state, wherein the delivery apparatus comprises an outer sheath configured to retain the prosthetic device in the radially compressed state when delivery the prosthetic device to the target location.

Example 95. The assembly of any example herein, particularly any one of examples 85-92, wherein the delivery apparatus comprises an actuator coupled to an expansion mechanism of the frame, wherein actuation of the actuator is configured to radially expand the prosthetic device from the radially compressed state to the radially expanded state.

Example 96. A method of assembling a prosthetic device, comprising: preparing an annular frame comprising: a row of outflow struts defining an outflow end of the annular frame; a row of inflow struts defining an inflow end of the annular frame; a row of interconnected struts circumferentially extending between the row of outflow struts and the row of inflow struts; and a plurality of axial frame members comprising respective first ends and second ends, the first ends being connected to the row of outflow struts and the second ends being connected to the row of interconnected struts; and bridging the plurality of axial frame members with a plurality of support members so that the support members circumferentially extend between the row of outflow struts and the row of interconnected struts, wherein each outflow strut comprises two angled strut portions interconnected by an apex portion, wherein the support members are narrower than the axial frame members, the angled strut portions of outflow struts, and the row of interconnected struts.

Example 97. The method of any example herein, particular example 96, wherein the bridging comprises connecting the plurality of support members to the plurality of axial frame members at connecting points that are axially located between the first ends and the second ends.

Example 98. The method of any example herein, particularly any one of examples 96-97, further comprising attaching a leaflet structure to the annular frame, wherein the leaflet structure is configured to permit blood flow from the inflow end to the outflow end and block blood fluid flow from the outflow end to the inflow end.

Example 99. The method of any example herein, particular example 98, wherein attaching the leaflet structure to the annular frame comprises connecting the leaflet structure to an inner skirt, and attaching the inner skirt to an inner surface of the annular frame.

Example 100. The method of any example herein, particularly any one of examples 98-99, wherein attaching the leaflet structure to the annular frame comprises attaching a plurality of commissures of the leaflet structure to respective commissure supports of the annular frame, wherein the commissure supports are selected members of the axial frame members.

Example 101. The method of any example herein, particularly any one of examples 96-100, further comprising attaching an outer skirt to an outer surface of the annular frame.

Example 102. The method of any example herein, particular example 101, wherein attaching the outer skirt to the outer surface of the annular frame comprises attaching an outflow edge portion of the outer skirt to the plurality of support members.

Example 103. The method of any example herein, particularly any one of examples 101-102, wherein attaching the outer skirt to the outer surface of the annular frame comprises attaching the outflow edge portion of the outer skirt to the axial frame members.

Example 104. The method of any example herein, particularly any one of examples 96-103, further comprising fully annealing the plurality of support members.

Example 105. A method of assembling a prosthetic device, comprising: preparing an annular frame comprising: a row of outflow struts defining an outflow end of the annular frame; a row of inflow struts defining an inflow end of the annular frame; a row of interconnected struts circumferentially extending between the row of outflow struts and the row of inflow struts; a plurality of axial frame members comprising respective first ends and second ends, the first ends being connected to the row of outflow struts and the second ends being connected to the row of interconnected struts; and a plurality of support members connecting the plurality of axial frame members and extending between the row of outflow struts and the row of interconnected struts; and attaching an outflow skirt to an outer surface of the annular frame, wherein the attaching comprises connecting an outflow edge portion of the outer skirt to the plurality of support members, wherein each outflow strut comprises two angled strut portions interconnected by an apex portion, wherein the support members are narrower than the axial frame members, the angled strut portions of the outflow struts, and the row of interconnected struts.

Example 106. A method, comprising: delivering a prosthetic device in a radially compressed state to a target location; and radially expanding the prosthetic device to a radially expanded state, wherein the prosthetic device comprises a radially expandable and compressible frame, wherein the frame comprises: a row of outflow struts defining an outflow end of the frame; a row of inflow struts defining an inflow end of the frame; a row of interconnected struts circumferentially extending between the row of outflow struts and the row of inflow struts; a plurality of axial frame members comprising respective first ends and second ends, the first ends being connected to the row of outflow struts and the second ends being connected to the row of interconnected struts; and a plurality of support members connecting the plurality of axial frame members and extending between the row of outflow struts and the row of interconnected struts, wherein each outflow strut comprises two angled strut portions interconnected by an apex portion, wherein the support members are narrower than the axial frame members, the angled strut portions of outflow struts, and the row of interconnected struts.

Example 107. A prosthetic valve, comprising: an annular frame that is movable between a radially compressed configuration and a radially expanded configuration, wherein the frame has an inflow end and an outflow end; and a leaflet structure positioned within the frame and configured to permit blood flow from the inflow end to the outflow end and block blood fluid flow from the outflow end to the inflow end, wherein the frame comprises: a first row of angled struts defining the outflow end; a second row of angled struts upstream of the first row of angled struts; a third row of angled struts upstream of the second row of angled struts; and a plurality of axial frame members comprising respective first ends and second ends, the first ends being connected to the second row of angled struts and the second ends being connected to the third row of angled struts; wherein the plurality of axial frame members comprise a plurality of axially extending commissure supports and one or more axial posts located between any two immediately adjacent commissure supports, wherein each commissure support is configured to support a corresponding commissure of the leaflet structure.

Example 108. The prosthetic valve of any example herein, particular example 107, wherein there are two or more angled struts in the second row connecting each pair of adjacent axial frame members.

Example 109. The prosthetic valve of any example herein, particularly any one of examples 107-108, wherein the second row of angled struts are joined together at alternating upper ends and lower ends, wherein the upper ends are axially located between the lower ends and the outflow end, wherein the first row of angled struts connect the second row of angled struts at the upper ends, wherein the plurality of axial frame members are connected to the lower ends.

Example 110. The prosthetic valve of any example herein, particularly any one of examples 107-108, wherein the second row of angled struts are joined together at alternating upper ends and lower ends, wherein the upper ends are axially located between the lower ends and the outflow end, wherein the first row of angled struts connect the second row of angled struts at the upper ends, wherein the plurality of axial frame members are connected to the upper ends.

Example 111. The prosthetic valve of any example herein, particularly any one of examples 107-110, wherein the first row of angled struts are narrower than the second row of angled struts.

Example 112. The prosthetic valve of any example herein, particularly any one of examples 107-111, wherein the second row of angled struts are wider than the third row of angled struts.

Example 113. The prosthetic valve of any example herein, particularly any one of examples 107-112, wherein the first row of angled struts and the second row of angled struts define a plurality of first cells of the frame, wherein the second row of angled struts, the third row of angled struts, and the plurality of axial frame members define a plurality of second cells of the frame, wherein the first cells are smaller than the second cells when the frame is in the radially expanded configuration.

Example 114. The prosthetic valve of any example herein, particular example 113, wherein the first cells have a diamond shape when the frame is in the radially expanded configuration.

Example 115. The prosthetic valve of any example herein, particularly any one of examples 113-114, wherein the number of the first cells and the number of second cells have a 1:1 ratio.

Example 116. The prosthetic valve of any example herein, particularly any one of examples 113-114, wherein the number of the first cells and the number of second cells have a M:1 ratio, wherein M is greater than 1.

Example 117. The prosthetic valve of any example herein, particular example 116, wherein M equals to 2.

Example 118. The prosthetic valve of any example herein, particularly any one of examples 113-117, wherein the number of second cells is 9.

Example 119. The prosthetic valve of any example herein, particularly any one of examples 113-118, further comprising a fourth row of angled struts upstream of the third row of angled struts, wherein the third row of angled struts and the fourth row of angled struts define a plurality of third cells of the frame, wherein the third cells are smaller than the second cells when the frame is in the radially expanded configuration.

Example 120. The prosthetic valve of any example herein, particular example 119, wherein the third cells are larger than the first cells when the frame is in the radially expanded configuration.

Example 121. The prosthetic valve of any example herein, particularly any one of examples 119-120, wherein the number of second cells is equal to the number of third cells.

Example 122. The prosthetic valve of any example herein, particularly any one of examples 119-121, further comprising a fifth row of angled struts upstream of the fourth row of angled struts, wherein the fifth row of angled struts define the inflow end.

Example 123. The prosthetic valve of any example herein, particularly any one of examples 107-114, wherein each of the first and second rows of angled struts comprise at least four or more angled struts extending between adjacent axial frame members

Example 124. A prosthetic valve, comprising: an annular frame that is movable between a radially compressed configuration and a radially expanded configuration, wherein the frame has an inflow end and an outflow end; and a leaflet structure positioned within the frame and configured to permit blood flow from the inflow end to the outflow end and block blood fluid flow from the outflow end to the inflow end, wherein the frame comprises: a first row of angled struts defining the outflow end; a second row of angled struts upstream of the first row of angled struts; a third row of angled struts upstream of the second row of angled struts; a fourth row of angled struts upstream of the third row of angled struts; and a plurality of axial frame members comprising respective first ends and second ends, the first ends being connected to the second row of angled struts and the second ends being connected to the third row of angled struts; wherein the first row of angled struts and the second row of angled struts define a plurality of first cells of the frame, wherein the second row of angled struts, the third row of angled struts, and the plurality of axial frame members define a plurality of second cells of the frame, wherein the third row of angled struts and the fourth row of angled struts define a plurality of third cells of the frame, wherein the number of first cells is more than the number of second cells, wherein the number of second cells equals the number of third cells.

Example 125. The prosthetic valve of any example herein, particular example 124, wherein the plurality of axial frame members comprise a plurality of axially extending commissure supports and one or more axial posts located between any two immediately adjacent commissure supports, wherein each commissure support is configured to support a corresponding commissure of the leaflet structure.

Example 126. The prosthetic valve of any example herein, particularly any one of examples 124-125, wherein the number of first cells is 18 and the number of second cells is 9.

Example 127. The prosthetic valve of any example herein, particularly any one of examples 124-126, wherein the first cells are smaller than the third cells and the third cells are smaller than the second cells when the frame is in the radially expanded configuration.

Example 128. An annular frame movable between a radially compressed configuration and a radially expanded configuration, the annular frame comprising: a first row of angled struts defining the outflow end; a second row of angled struts upstream of the first row of angled struts; a third row of angled struts upstream of the second row of angled struts; a fourth row of angled struts upstream of the third row of angled struts; and a plurality of axial frame members comprising respective first ends and second ends, the first ends being connected to the second row of angled struts and the second ends being connected to the third row of angled struts; wherein the first row of angled struts and the second row of angled struts define a plurality of first cells of the frame, wherein the second row of angled struts, the third row of angled struts, and the plurality of axial frame members define a plurality of second cells of the frame, wherein the third row of angled struts and the fourth row of angled struts define a plurality of third cells of the frame, wherein the first cells are smaller than the third cells and the third cells are smaller than the second cells when the frame is in the radially expanded configuration.

Example 129. The annular frame of any example herein, particular example 128, wherein the number of first cells is a multiple of the number of second cells, wherein the number of second cells equals the number of third cells.

Example 130. The annular frame of any example herein, particularly any one of examples 128-129, wherein the first row of angled struts forms a plurality of arcs when the frame is in the radially expanded configuration.

Example 131. The annular frame of any example herein, particularly any one of examples 128-130, wherein the plurality of axial frame members comprise a plurality of axially extending commissure supports and one or more axial posts located between any two immediately adjacent commissure supports, wherein each commissure support is configured to support a corresponding commissure of a leaflet structure mounted within the annular frame.

Example 132. A prosthetic valve comprising an annular frame that is movable between a radially compressed configuration and a radially expanded configuration, wherein the frame has an inflow end and an outflow end; and a leaflet structure positioned within the frame and configured to permit blood flow from the inflow end to the outflow end and block blood fluid flow from the outflow end to the inflow end, wherein the frame comprises: a first row of angled struts defining the outflow end; a second row of angled struts upstream of the first row of angled struts; a third row of angled struts upstream of the second row of angled struts; a plurality of axial frame members bridging the second row of angled struts and the third row of angled struts; wherein the plurality of axial frame members comprise a plurality of axially extending commissure supports and one or more axial posts located between any two immediately adjacent commissure supports, wherein the second row of angled struts, the third row of angled struts, and the plurality of axial frame members define a plurality of outflow cells, wherein the frame further comprises one or more axial support struts dividing at least some of the outflow cells in halves.

Example 133. The prosthetic valve of any example herein, particular example 132, wherein the one or more axial support struts are narrower than the plurality of axial frame members.

Example 134. The prosthetic valve of any example herein, particular any one of examples 132-133, wherein the one or more axial support struts are longer than the plurality of axial frame members.

Example 135. A prosthetic valve comprising a frame, the frame comprising: an inflow end; an outflow end; a row of outflow struts defining the outflow end; a row of inflow struts defining the inflow end; an intermediate row of struts located axially between the row of outflow struts and the row of inflow struts; a plurality of axial frame members comprising respective first ends and second ends, the first ends being connected to the row of outflow struts and the second ends being connected to the intermediate row of struts; and a circumferentially extending row of support members located axially between the row of outflow struts and the intermediate row of struts, the support members connecting the plurality of axial frame members, wherein each outflow strut comprises two angled strut portions interconnected by an apex portion, a plurality of axial support struts having first ends connected to respective apex portions of the outflow struts and second ends connected to respective joint portions of the support members.

Example 136. The prosthetic valve of any examples herein, particular example 135, wherein the support members are narrower than the axial frame members, the angled strut portions of the outflow struts, and the struts of the intermediate row of struts.

Example 137. A prosthetic valve, comprising: an annular frame that is radially expandable and compressible; and wherein the annular frame comprises: a row of outflow struts defining an outflow end of the frame; a row of inflow struts defining an inflow end of the frame; an intermediate row of angled struts located axially between the row of outflow struts and the row of inflow struts; a plurality of axial frame members bridging the row of outflow struts and the intermediate row of angled struts; and a plurality of support structures connecting the plurality of axial frame members and the intermediate row of angled struts, wherein each support structure comprises at least two angled support arms connecting two immediately adjacent axial frame members and at least one axial support member bridging the at least two angled support arms and the intermediate row of angle struts.

Example 138. The prosthetic valve of any examples herein, particular example 137, wherein the angled support arms are narrower than the outflow struts and the inflow struts.

Example 139. The prosthetic valve of any example herein, particular any one of examples 137-138, wherein the axial support members are narrower than the axial frame members.

Example 140. The prosthetic valve of any example herein, particular any one of examples 137-139, wherein the angled support arms are narrower than the axial support members.

Example 141. The prosthetic valve of any example herein, particular any one of examples 137-140, wherein each axial frame member comprises a first end connected to the row of outflow struts, a second end connected to the intermediate row of angled struts, and a connecting point where two angled support arms of two adjacent support structures located on both sides of the axial frame member are connected to the axial frame member.

Example 142. The prosthetic valve of any examples herein, particular example 141, wherein for each axial frame member, the first end is the connecting point.

Example 143. The prosthetic valve of any examples herein, particular example 141, wherein for each axial frame member, the connecting point is axially spaced away from the first end.

Example 144. The prosthetic valve of any examples herein, particular example 143, wherein for each axial frame member, the connecting point is closer to the first end than the second end.

Example 145. The prosthetic valve of any example herein, particular any one of examples 137-144, further comprising an outer skirt disposed on an outer surface of the annular frame, wherein the outer skirt is attached to the angled support arms of each support structure.

Example 146. The prosthetic valve of any example herein, particular any one of examples 137-145, wherein each support structure comprises exactly two angled support arms and exactly one axial support member, wherein the two angled support arms and the one axial support member define a Y-shaped configuration.

Example 147. The prosthetic valve of any examples herein, particular example 146, wherein the intermediate row of angled struts are joined together at alternating upper ends and lower ends, the upper ends being closer to the outflow end than the lower ends, wherein the axial support members of the support structures are connected to corresponding lower ends of the intermediate row of angled struts.

Example 148. The prosthetic valve of any examples herein, particular example 147, wherein the row of outflow struts, the intermediate row of angled struts, and the plurality of axial frame members define a plurality of outflow cells, wherein each support structure divides a corresponding outflow cell into three sub-cells.

Example 149. The prosthetic valve of any examples herein, particular example 148, wherein the three sub-cells comprises an upper sub-cell and two lower sub-cells, wherein the upper sub-cell has a width that is twice that of each lower sub-cell.

Example 150. The prosthetic valve of any example herein, particular any one of examples 148-149, wherein the upper sub-cell is six-sided or four-sided.

Example 151. The prosthetic valve of any example herein, particular any one of examples 148-150, wherein each of the lower sub-cell has a parallelogram shape.

Example 152. The prosthetic valve of any example herein, particular any one of examples 146-151, wherein the intermediate row of angled struts is the only row of angled struts extending between the row of outflow struts and the row of inflow struts.

Example 153. The prosthetic valve of any example herein, particular any one of examples 146-151, wherein the intermediate row of angled struts is a first intermediate row of angled struts, wherein there are one or more additional intermediate row of angled struts between the first intermediate row of angled struts and the row of inflow struts.

Example 154. The prosthetic valve of any examples herein, particular example 153, wherein there are exactly two intermediate rows of angled struts between the row of outflow struts and the row of inflow struts.

Example 155. The prosthetic valve of any examples herein, particular example 153, wherein there are exactly three intermediate rows of angled struts between the row of outflow struts and the row of inflow struts.

Example 156. The prosthetic valve of any example herein, particular any one of examples 146-151, wherein the axial support members of the support structures terminate at the corresponding lower ends of the intermediate row of angled struts.

Example 157. The prosthetic valve of any example herein, particular any one of examples 146-151, wherein the axial support members of the support structures further bridge the intermediate row of angled struts and the row of inflow struts.

Example 158. The prosthetic valve of any examples herein, particular example 157, wherein the row of inflow struts define a plurality of upper ends downstream of the inflow end, wherein the axial support members of the support structures connect the lower ends of the intermediate row of angled struts to the corresponding upper ends of the row of inflow struts.

Example 159. The prosthetic valve of any examples herein, particular example 158, wherein the number of apices defined by the outflow struts and the number of apices defined by the inflow struts has a 1:2 ratio.

Example 160. The prosthetic valve of any example herein, particular any one of examples 146-151, wherein the plurality of support structures are first support structures, wherein the annular frame further comprises a plurality of second support structures, wherein each second support structure comprises two angled support arms and the one axial support member that define an inverted Y-shaped configuration.

Example 161. The prosthetic valve of any examples herein, particular example 160, wherein the intermediate row of angled struts is a first intermediate row of angled struts and the plurality of axial frame members are first axial frame members, wherein the annular frame further comprises a second intermediate row of angled struts upstream of the first intermediate row of angled struts and a plurality of second axial frame members bridging the second intermediate row of angled struts and the row of inflow struts.

Example 162. The prosthetic valve of any examples herein, particular example 161, wherein the axial support members of the first support structures are connected to axial support members of the second support structures at junctions between the first and second intermediate rows of angled struts such that each first support structure mirrors a corresponding second support structure.

Example 163. The prosthetic valve of any example herein, particular any one of examples 161-162, wherein for each second support structure, the two angled support arms connect two immediately adjacent second axial frame members and the axial support member bridges the two angled support arms and the second intermediate row of angle struts.

Example 164. The prosthetic valve of any example herein, particular any one of examples 137-144, wherein each support structure comprises exactly four angled support arms that are interconnected to define a W-shaped configuration, wherein the four angled support arms comprise two side support arms respectively connected to two adjacent axial frame members and two middle support arms positioned between the two side support arms.

Example 165. The prosthetic valve of any examples herein, particular example 164, wherein there are exactly six axial frame members.

Example 166. The prosthetic valve of any example herein, particular any one of examples 164-165, wherein the intermediate row of angled struts are joined together at alternating upper ends and lower ends, wherein each support structure comprises a middle axial support member connecting a joint of the two middle support arms and a corresponding upper end of the intermediate row of angled struts.

Example 167. The prosthetic valve of any examples herein, particular example 166, wherein the row of outflow struts, the intermediate row of angled struts, and the plurality of axial frame members define a plurality of outflow cells, wherein each support structure divides a corresponding outflow cell into an upper sub-cell and two lower sub-cells.

Example 168. The prosthetic valve of any examples herein, particular example 167, wherein each lower sub-cell is six-sided, and the upper sub-cell has a width that is twice that of each lower sub-cell.

Example 169. The prosthetic valve of any examples herein, particular example 166, wherein each support structure further comprises two side axial support members located on opposite sides of the middle support member, wherein each side axial support member connects one of the lower ends of the intermediate row of angled struts and a joint formed between a side support arm and an adjacent middle support arm.

Example 170. The prosthetic valve of any examples herein, particular example 169, wherein the row of outflow struts, the intermediate row of angled struts, and the plurality of axial frame members define a plurality of outflow cells, wherein each support structure divides a corresponding outflow cell into an upper sub-cell and four lower sub-cells.

Example 171. The prosthetic valve of any examples herein, particular example 170, wherein each of the lower sub-cell has a width that is one quarter of the width of the upper sub-cell.

Example 172. The prosthetic valve of any example herein, particular any one of examples 170-171, wherein each of the lower sub-cell has a parallelogram shape.

Example 173. A prosthetic valve, comprising: an annular frame that is radially expandable and compressible, wherein the annular frame comprises: a row of outflow struts defining an outflow end of the frame; a row of inflow struts defining an inflow end of the frame; first and second intermediate rows of angled struts located axially between the row of outflow struts and the row of inflow struts, the second intermediate row of angled struts being upstream of the first intermediate row of angled struts; a plurality of axial frame members bridging the row of outflow struts and the first intermediate row of angled struts; and a plurality of support structures connecting the plurality of axial frame members and the first and second intermediate rows of angled struts, wherein each support structure comprises at least two angled support arms connecting two immediately adjacent axial frame members and two or more axial support members bridging the at least two angled support arms and the first and second intermediate rows of angle struts.

Example 174. The prosthetic valve of any examples herein, particular example 173, wherein the angled support arms are narrower than the outflow struts and the inflow struts.

Example 175. The prosthetic valve of any example herein, particular any one of examples 173-174, wherein the axial support members are narrower than the axial frame members.

Example 176. The prosthetic valve of any example herein, particular any one of examples 173-175, further comprising an outer skirt disposed on an outer surface of the annular frame, wherein the outer skirt is connected to at least parts of the support structures.

Example 177. The prosthetic valve of any example herein, particular any one of examples 173-176, wherein each axial frame member comprises a first end connected to the row of outflow struts, a second end connected to the first intermediate row of angled struts, and a connecting point where two angled support arms of two adjacent support structures located on both sides of the axial frame member are connected to the axial frame member.

Example 178. The prosthetic valve of any examples herein, particular example 177, wherein for each axial frame member, the first end is the connecting point.

Example 179. The prosthetic valve of any examples herein, particular example 177, wherein for each axial frame member, the connecting point is axially spaced away from the first end.

Example 180. The prosthetic valve of any example herein, particular any one of examples 177-179, wherein the second end of each axial frame member is connected to two angled struts in the first intermediate row of angled struts that are not directly connected to any other angled struts in the first intermediate row of angled struts.

Example 181. The prosthetic valve of any example herein, particular any one of examples 177-180, wherein the second intermediate row of angled struts are joined together at alternating upper ends and lower ends, wherein each angled strut in the first intermediate row of angled struts is connected to one of the upper ends of the second intermediate row of angled struts.

Example 182. The prosthetic valve of any examples herein, particular example 181, wherein each support structure comprises exactly two angled support arms, one middle axial support member, and two side axial support members, wherein the middle axial support member has an upper end connected to a joint of the two angled support arms, wherein the two side axial support members have respective upper ends connected to corresponding mid-points of the two angled support arms.

Example 183. The prosthetic valve of any examples herein, particular example 182, wherein each support structure further comprises one connecting support member which connects the mid-points of the two angled support arms.

Example 184. The prosthetic valve of any example herein, particular any one of examples 182-183, wherein for each support structure, the middle axial support member has a lower end connected to one of the lower ends of the second intermediate row of angled struts, wherein the two side axial support members have respective lower ends connected to corresponding upper ends of the second intermediate row of angled struts.

Example 185. The prosthetic valve of any example herein, particular any one of examples 182-184, wherein the row of outflow struts, the first and second intermediate rows of angled struts, and the plurality of axial frame members define a plurality of outflow cells, wherein each support structure divides a corresponding outflow cell into a plurality of sub-cells, wherein the plurality of sub-cells comprise a row of four sub-cells each of which has a parallelogram shape.

Example 186. The prosthetic valve of any examples herein, particular example 181, wherein each support structure comprises eight angled support arms and four axial support members, wherein each axial support member has an upper end connected to a corresponding joint of two angled support arms and a lower end connected to a corresponding upper end of the second intermediate row of angled struts.

Example 187. The prosthetic valve of any examples herein, particular example 186, wherein the row of outflow struts, the first and second intermediate rows of angled struts, and the plurality of axial frame members define a plurality of outflow cells, wherein each support structure divides a corresponding outflow cell into a plurality of sub-cells, wherein the plurality of sub-cells comprise a row of five sub-cells, wherein the row of five sub-cells comprise three six-sided sub-cells located between two parallelogram-shaped sub-cells.

Example 188. A method comprising sterilizing prosthetic valves, frames, and/or assemblies of any examples herein, particularly any one of examples 1-95 and 107-187.

The features described herein with regard to any example can be combined with other features described in any one or more of the other examples, unless otherwise stated. For example, any one or more of the features of one frame or prosthetic valve can be combined with any one or more features of another frame or prosthetic valve.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A prosthetic valve, comprising:
an annular frame that is radially collapsible to a collapsed configuration and radially expandable to an expanded configuration; and
an outer skirt disposed on an outer surface of the annular frame,
wherein the annular frame comprises:
an inflow end;
an outflow end;
a first row of angled struts defining the outflow end;
a second row of angled struts being closer to the inflow end than the first row of angled struts;
a plurality of axial frame members bridging the first row of angled struts and the second row of angled struts; and
a plurality of support members connecting the plurality of axial frame members,
wherein the support members are narrower than the axial frame members and the first and second rows of angled struts,
wherein the outer skirt is connected to the support members.

2. The prosthetic valve of claim 1, wherein the annular frame comprises at least four rows of angled struts.

3. The prosthetic valve of claim 1, wherein each support member comprises two arm portions and a joint portion connecting the two arm portions, wherein each support member is configured to fold at the joint portion when the annular frame moves from the expanded configuration to the collapsed configuration.

4. The prosthetic valve of claim 3, wherein the joint portion of each support member is configured to move axially toward the inflow end when the annular frame moves from the expanded configuration to the collapsed configuration.

5. The prosthetic valve of claim 4, wherein the joint portion of each support member is configured to move axially toward the outflow end when the annular frame moves from the expanded configuration to the collapsed configuration.

6. A prosthetic valve, comprising:
an annular frame that is radially expandable and compressible; and
an outer skirt disposed on an outer surface of the annular frame,
wherein the annular frame comprises:
an inflow end;
an outflow end;
a first row of angled struts defining the outflow end and a second row of angled struts upstream of the first row of angled struts;
a plurality of axial frame members comprising respective first ends and second ends, the first ends being connected to the first row of angled struts and the second ends being connected to the second row of angled struts; and
a plurality of support members connecting the plurality of axial frame members at connecting points located between the first ends and the second ends,
wherein the outer skirt is connected to the support members.

7. The prosthetic valve of claim 6, further comprising a plurality of leaflets positioned within the annular frame and configured to permit blood flow from the inflow end to the outflow end and block blood fluid flow from the outflow end to the inflow end.

8. The prosthetic valve of claim 7, wherein the plurality of axial frame members comprises a plurality of axially extending commissure supports and one or more axial posts located between any two immediately adjacent commissure supports, wherein each commissure support comprises a commissure window configured to receive a corresponding commissure of two adjacent leaflets.

9. The prosthetic valve of claim 8, wherein the plurality of axial frame members comprise three axially extending commissure supports and exactly two axial posts located between any two immediately adjacent commissure supports.

10. The prosthetic valve of claim 8, wherein at least some of the support members connect to the commissure supports at the connecting points located between the first ends and the commissure windows.

11. The prosthetic valve of claim 8, wherein at least some of the support members connect to the commissure supports at the connecting points located between the second ends and the commissure windows.

12. The prosthetic valve of claim 6, wherein the support members are narrower than the axial frame members and the first and second rows of angled struts.

13. The prosthetic valve of claim 6, wherein the annular frame further comprises a third row of angled struts upstream of the second rows of angled struts and a fourth rows of angled struts upstream of the third rows of angled struts, wherein the fourth row of angled struts defines the inflow end.

14. The prosthetic valve of claim 13, wherein second row of angled struts and the third row of angled struts have the same width, and the first row of angled struts and the fourth row of angled struts have the same width.

15. The prosthetic valve of claim 13, wherein the axial frame members are wider than any of the first, second, third, and fourth rows of angled struts.

16. An annular frame that is radially expandable and compressible, the annular frame comprising:
a row of first end struts defining a first terminal end of the annular frame;
a row of second end struts defining a second terminal end of the annular frame;
a plurality of interconnected struts arranged in one or more rows circumferentially extending between the row of first end struts and the row of second end struts;
a plurality of axial frame members comprising respective first ends and second ends, the first ends being connected to the row of first end struts and the second ends being connected to a first row of interconnected struts; and
a plurality of support members connecting the plurality of axial frame members at connecting points that are axially located between the first ends and the second ends.

17. The annular frame of claim 16, wherein connecting points are axially closer to the first ends than a midpoint between the first ends and the second ends.

18. The annular frame of claim 16, wherein a midpoint between the first ends and the second ends is axially closer to the first ends than the connecting points.

19. The annular frame of claim 16, wherein each support member comprises two angled arm portions and a joint portion connecting the two angled arm portions.

20. The annular frame of claim 19, wherein the joint portions of the support members are axially closer to the second ends than the connecting points when the annular frame is radially expanded.

21. The annular frame of claim 19, wherein the joint portions of the support members are axially closer to the first ends than the connecting points when the annular frame is radially expanded.

22. The annular frame of claim 19, wherein the joint portions of the support members are axially located between the first ends and the second ends of the axial frame members when the annular frame is radially expanded.

23. The annular frame of claim 19, wherein the joint portions are axially aligned with the first ends of the axial frame members when the annular frame is radially expanded.
